(19) United States
(12) Patent Application Publication
(10) Pub. No.: US 2021/0198607 A1
(43) Pub. Date: Jul. 1, 2021

(54) MULTICOMPARTMENT MICROFLUIDIC BIOREACTORS, CYLINDRICAL ROTARY VALVES AND APPLICATIONS OF SAME

(71) Applicant: VANDERBILT UNIVERSITY, Nashville, TN (US)

(72) Inventors: John P. WIKSWO, Brentwood, TN (US); Eric C. SPIVEY, Nashville, TN (US); David K. SCHAFFER, Nashville, TN (US); Ronald S. REISERER, Nashville, TN (US); Kevin T. SEALE, Nashville, TN (US); Frank E. BLOCK, III, Potomac, MD (US)

(21) Appl. No.: 17/057,267

(22) PCT Filed: May 29, 2019
(86) PCT No.: PCT/US2019/034285

§ 371 Date: Nov. 20, 2020

Related U.S. Application Data
(63) Continuation-in-part of application No. 16/012,900, filed on Jun. 20, 2018, now Pat. No. 10,577,574.
Application No. 16/012,900 is a division of application No. 15/191,092, filed on Jun. 23, 2016, now Pat. No. 10,023,832.
Application No. 15/191,092 is a continuation-in-part of application No. 13/877,925, filed on Jul. 16, 2013, now abandoned.
Application No. 15/191,092 is a continuation-in-part of application No. 14/363,074, filed on Jun. 5, 2014, now Pat. No. 10,078,075.
Application No. 15/191,092 is a continuation-in-part of application No. 14/646,300, filed on May 20, 2015, now Pat. No. 9,874,285.
Application No. 15/191,092 is a continuation-in-part of application No. 14/651,174, filed on Jun. 10, 2015, now Pat. No. 9,618,129.
Application No. PCT/US2019/034285 is a continuation-in-part of application No. 15/776,524, filed on May 16, 2018, now Pat. No. 10,532,354.
Application No. 15/776,524 is a continuation-in-part of application No. 13/877,925, filed on Jul. 16, 2013, now abandoned.
Application No. 13/877,925 is a continuation-in-part of application No. 14/363,074, filed on Jun. 5, 2014, now Pat. No. 10,078,075.
Application No. 14/363,074 is a continuation-in-part of application No. 14/646,300, filed on May 20, 2015, now Pat. No. 9,874,285.
Application No. 14/646,300 is a continuation-in-part of application No. 14/651,174, filed on Jun. 10, 2015, now Pat. No. 9,618,129.
Application No. 14/651,174 is a continuation-in-part of application No. 15/191,092, filed on Jun. 23, 2016, now Pat. No. 10,023,832.
(60) Provisional application No. 62/677,468, filed on May 29, 2018.
(60) Provisional application No. 62/183,571, filed on Jun. 23, 2015.
(60) Provisional application No. 62/193,029, filed on Jul. 15, 2015.
(60) Provisional application No. 62/276,047, filed on Jan. 7, 2016.
(60) Provisional application No. 62/295,306, filed on Feb. 15, 2016.
(60) Provisional application No. 61/390,982, filed on Oct. 7, 2010.
(60) Provisional application No. 61/569,145, filed on Dec. 9, 2011.
(60) Provisional application No. 61/697,204, filed on Sep. 5, 2012.
(60) Provisional application No. 61/717,441, filed on Oct. 23, 2012.
(60) Provisional application No. 61/729,149, filed on Nov. 21, 2012.
(60) Provisional application No. 61/808,455, filed on Apr. 4, 2013.
(60) Provisional application No. 61/822,081, filed on May 10, 2013.
(60) Provisional application No. 61/808,455, filed on Apr. 4, 2013.
(60) Provisional application No. 61/822,081, filed on May 10, 2013.
(60) Provisional application No. 62/259,327, filed on Nov. 24, 2015.

(51) Int. Cl. C12M 3/06; A01N 1/02; C12M 3/00; C12M 1/00; C12M 1/32; C12M 1/36; F04B 43/00; B01L 3/00

(52) U.S. Cl. CPC C12M 23/16; A01N 1/0247; C12M 23/44; C12M 29/00; B01L 3/502715; C12M 41/48; F04B 43/0045; C12M 23/58; B01L 3/5027; C12M 23/12

(57) ABSTRACT

One aspect of the invention provides a multichamber bioreactor. The multichamber bioreactor includes multiple planar layers stacked on each other defining at least one chamber and a clamping mechanism. The clamping mechanism includes a housing and retaining means received in the housing and configured to generate a controlled and uniform pressure to secure the stacked multiple planar layers in the housing. Each chamber is implemented from a separate fluidic layer, with each fluidic layer having ports and valves independent of the other layers. The micro fluidic ports can be actuated through a micro fluidic interconnect system utilizing rotary cylinder valves.

### CROSS-REFERENCE TO RELATED PATENT APPLICATIONS

This application claims priority to and the benefit of U.S. Provisional Patent Application Ser. No. 62/677,468, filed May 29, 2018.

This application is also a continuation-in-part application of U.S. Patent Application Ser. No. 16/012,900, filed Jun. 20, 2018, which is a divisional application of U.S. patent application Ser. No. 15/191,092 (the '092 application), filed Jun. 23, 2016, now U.S. Pat. No. 10,023,832, which claims priority to and the benefit of U.S. Provisional Patent Application Ser. Nos. 62/183,571, 62/193,029, 62/276,047 and 62/295,306, filed Jun. 23, 2015, Jul. 15, 2015, Jan. 7, 2016 and Feb. 15, 2016, respectively. The '092 application is also a continuation-in-part application of U.S. patent spplication Ser. Nos. 13/877,925 (the '925 application), 14/363,074 (the '074 application), 14/646,300 (the '300 application) and 14/651,174 (the '174 application), filed Jul. 16, 2013, Jun. 5, 2014, May 20, 2015 and Jun. 10, 2015, respectively. The '925 application, now abandoned, is a national stage entry of PCT Application Serial No. PCT/US2011/055432, filed Oct. 7, 2011, which claims priority to and the benefit of U.S. Provisional Patent Application Ser. No. 61/390,982, filed Oct. 7, 2010. The '074 application, now U.S. Pat. No. 10,078,075, is a national stage entry of PCT Application Serial No. PCT/US2012/068771, filed Dec. 10, 2012, which claims priority to and the benefit of U.S. Provisional Patent Application Ser. Nos. 61/569,145, 61/697,204 and 61/717,441, filed Dec. 9, 2011, Sep. 5, 2012 and Oct. 23, 2012, respectively. The '300 application, now U.S. Pat. No. 9,874,285, is a national stage entry of PCT Application Serial No. PCT/US2013/071026, filed Nov. 20, 2013, which claims priority to and the benefit of U.S. Provisional Patent Application Ser. Nos. 61/729,149, 61/808,455, and 61/822,081, filed Nov. 21, 2012, Apr. 4, 2013 and May 10, 2013, respectively. The '174 application, now U.S. Pat. No. 9,618,129, is a national stage entry of PCT Application Serial No. PCT/US2013/071324, filed Nov. 21, 2013, which claims priority to and the benefit of U.S. Provisional Patent Application Ser. Nos. 61/808,455 and 61/822,081, filed Apr. 4, 2013 and May 10, 2013, respectively.

This application is also a continuation-in-part application of U.S. Patent Application Ser. No. 15/776,524, filed May 16, 2018, now allowed, which is a national stage entry of PCT Application Ser. No. PCT/US2016/063586 (the '586 application), filed Nov. 23, 2016, which claims priority to and the benefit of, U.S. Provisional Patent Application Ser. No. 62/259,327, filed Nov. 24, 2015. The '586 application is also a continuation-in-part application of U.S. patent application Ser. Nos. 13/877,925 (the '925 application), 14/363,074 (the '074 application), 14/646,300 (the '300 application), 14/651,174 (the '174 application) and 91,092 (the '092 application), filed Jul. 16, 2013, Jun. 5, 2014, May 20, 2015, Jun. 10, 2015 and Jun. 23, 2016, respectively.

Each of the above-identified applications is incorporated herein by reference in its entirety.

Some references, which may include patents, patent applications, and various publications, are cited and discussed in the description of the invention. The citation and/or discussion of such references is provided merely to clarify the description of the invention and is not an admission that any such reference is “prior art” to the invention described herein. All references cited and discussed in this specification are incorporated herein by reference in their entireties and to the same extent as if each reference was individually incorporated by reference. In terms of notation, hereinafter, [n] represents the nth reference cited in the reference list. For example, [3] represents the third reference cited in the reference list, namely, Wikswo et al., Engineering Challenges for Instrumenting and Controlling Integrated Organ-on-Chip Systems. IEEE Trans. Biomed. Eng., 60:682-690, 2013.

### STATEMENT AS TO RIGHTS UNDER FEDERALLY-SPONSORED RESEARCH

The invention was made with government support under Grant Nos. 5UG3TR002097-02 and 1U01TR002383-01 awarded by the National Center for Advancing Translational Sciences (NCATS), Contract No. CBMXCEL-XL1-2-001 awarded by the Defense Threat Reduction Agency (DTRA), and contract 2017-17081500003 awarded by the Intelligence Advanced Research Projects Activity (IARPA). The government has certain rights in the invention.

### FIELD OF THE INVENTION

The invention relates generally to microfluidic systems, and more particularly to multicompartment microfluidic bioreactors that can be readily assembled, disassembled, interconnected, and isolated and can utilize cylindrical rotary valves, and applications of the same.

### BACKGROUND INFORMATION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

There are a variety of means by which a microfluidic bioreactor can be sealed, i.e., assembled or disassembled in a manner that is reversable yet precludes leaks. For example, an upper plate can be compressed against a lower plate by a plurality of screws, as demonstrated by Shah et al. [1] and many others. The clamping force can be applied by a systems of levers and latches, such as disclosed by Brunswig, et al [2]. Alternatively, component layers can be irreversibly bonded to each other, for example as is done with plasma-activated polydimethylsiloxane (PDMS) microfluidic devices. Layers can be held together through autoadhesion, or by a vacuum channel around the periphery of the device. It is possible to create a single-chamber, multiple-layer, fluidic flow cell whose volume in some configurations is adjustable, for example for infra-red transmission spectroscopy (Specac), but these devices do not provide separate delivery and withdrawal fluidic access to more than one fluidic chamber.

In addition, there is also a need to seal the ports of a microfluidic bioreactor to allow connection of the bioreactor to perfusion systems or analytical devices, and also to disconnect them without introducing bubbles or contamination or losing fluid to the external environment. Some microfluidic bioreactors use external Luer-lock shut-off valves, but these valves are bulky and are not suitable for controlled sterilization of the interconnection or the elimination of bubbles. Other bioreactors have a means for controlled connection, but without valving, e.g., disclosed in U.S. Pat. No. 8,533,413 by Brunswig, et al. [2].

Therefore, a heretofore unaddressed need exists in the art to address the aforementioned deficiencies and inadequacies.

### SUMMARY OF THE INVENTION

In one aspect, the invention relates to a multichamber bioreactor comprising multiple planar layers stacked on each other defining at least one chamber. In one embodiment, each chamber is implemented from a separate fluidic layer, with each fluidic layer having ports and valves independent of the other layers.

In one embodiment, the multichamber bioreactor further comprises a clamping mechanism. The clamping mechanism comprises a housing and retaining means received in the housing and configured to generate a controlled and uniform pressure to secure the stacked multiple planar layers in the housing.

In one embodiment, the stacked multiple planar layers comprise an endothelial microfluidic disc; at least one somatic cell chamber layer disposed on the endothelial microfluidic disc; a microfluidic perfusion disc disposed on the at least one somatic cell chamber layer; a pressure plate disposed on the at least one somatic cell chamber layer; and at least one membrane, each member disposed between the endothelial microfluidic disc and the at least one somatic cell chamber layer, and between two adjacent somatic cell chamber layers when the at least one somatic cell chamber layer has two or more somatic cell chamber layers. The stacked multiple planar layers are placed on a base plate, received in the housing.

In one embodiment, when high shear flow is not required, the stacked multiple planar layers comprise two perfusion discs, but otherwise as described in the paragraph above. The exact layout and depths of the perfusion channels in any of the layers can be adjusted to match the particular needs of the bioreactor.

In one embodiment, each of the pressure plate and the microfluidic perfusion disc has a plurality of through holes defined therein, and aligned to each other in the stacked multiple planar layers, such that a plurality of tubes with flexible lengths is insertable into the through holes of the pressure plate and the microfluidic perfusion disc for connecting to an individual layer.

In one embodiment, the microfluidic perfusion disc has notches formed on its edge and tubing sockets protruded from the microfluidic perfusion disc for connecting a plurality of tubes to an individual layer; the somatic cell chamber layer has tubing sockets protruded from the somatic cell chamber layer for connecting the plurality of tubes to an individual layer; and the pressure plate has notches formed its edge, such that, as assembled, the tubing sockets of the somatic cell chamber layer are received in the tubing sockets of the microfluidic perfusion disc, which are in turn received in the notches of the pressure plate, so as to allow each upper layer to be inserted over a lower layer without having to thread the plurality of tubes through individual holes in each upper layer.

In one embodiment, the multichamber bioreactor further comprises a transwell adapter for accommodating the at least one somatic cell chamber layer to allow culture of cells independent of the bioreactor prior to insertion of, or after extraction of the at least one somatic cell chamber layer.

In one embodiment, the housing has a threaded inner surface, wherein the retaining means comprises at least one threaded retaining ring being operably threaded into the housing to secure the stacked multiple planar layers in the housing.

In one embodiment, the housing has slots formed in a wall of the housing, and wherein the retaining means comprises a retaining ring having pins radially protruded from a peripheral side of the retaining ring being operably fitted into the slots of the housing 401 to secure the stacked multiple planar layers in the housing. In one embodiment, the slots are L-shape slots.

In one embodiment, the retaining means comprises an expanding clamp pressing outwards against an inner surface of the housing to be held in place by force of friction between the sides of the expanding clamp and the inner surface of the housing.

In one embodiment, the housing includes an internally and externally threaded, notched crown housing, and wherein the retaining means comprises an externally-threaded retaining ring inside the crown; two internally threaded retaining rings outside the crown; a fluidic interface bottom support; a fluidic interface top support; and a microfluidic interface disposed between the fluidic interface bottom support and the fluidic interface top support, wherein the fluidic interface bottom and top supports are designed to provide mechanical support for insertion of tubes or ribbon connectors into the fluidic and to protect the fluidic during handling of the multichamber bioreactor.

In one embodiment, the microfluidic interface comprises conduits embossed on underside and vertical ports. In one embodiment, the two ports and channels furthest from an axis of the conduits are connected to an endothelial chamber of the endothelial microfluidic disc, and the four ports and channels closest to the axis are connected to a stromal cell chamber of the somatic cell chamber layer. In one embodiment, the ports and channels for one layer are directed towards one slot of the crown housing, and the ports for the other layer are directed to a different slot of the crown housing.

In one embodiment, the microfluidic interface further comprises shut-off valves coupled between the conduits and the ports.

In one embodiment, the multichamber bioreactor is operably connected to a perfusion controller, and/or a second, downstream multichamber bioreactor, by at least one connector assembly.

In one embodiment, the multichamber bioreactor further comprises valves integrated into input and output fluidic lines to enable sterilization and washing of open ports of the fluidic connections and elimination of bubbles during the connection process before the ports are connected to the bioreactor chambers and external support equipment, and eliminate leakage of fluid when disconnected.

In one embodiment, the multichamber bioreactor is insertable into a holder whose size is consistent with a standard well plate and whose individual chambers can be cultured separately prior to assembly, with a rotating or friction-fit retaining ring or rings within a structure with a hollow cylindrical region that aligns and holds components together without need for irreversible bonding or multiple fasteners or levers.

In one embodiment, the multichamber bioreactor is rapidly disassembled for imaging or analysis of cellular contents or for their interfacing to other systems and components. In one embodiment, the multichamber bioreactor is operable with an arbitrary number of chambers in a vertical stack without modifications to hardware.

In another aspect, the invention relates to a clamping device, comprising a housing; and retaining means received in the housing and configured to generate a controlled and uniform pressure to secure a layered, planar multi-chamber microfluidic bioreactor in the housing.

In one embodiment, the housing has a threaded inner surface, wherein the retaining means comprises at least one threaded retaining ring being operably threaded into the housing to secure the layered, planar multi-chamber microfluidic bioreactor in the housing.

In one embodiment, the housing has slots formed in a wall of the housing, and wherein the retaining means comprises a retaining ring having pins radially protruded from a peripheral side of the retaining ring being operably fitted into the slots of the housing to secure the layered, planar multi-chamber microfluidic bioreactor in the housing. In one embodiment, the slots are L-shape slots.

In one embodiment, the retaining means comprises an expanding clamp pressing outwards against an inner surface of the housing to be held in place by force of friction between the sides of the expanding clamp and the inner surface of the housing.

In one embodiment, the housing includes an internally and externally threaded, notched crown housing, and wherein the retaining means comprises an externally-threaded retaining ring, and two internally threaded retaining rings.

In yet another aspect, the invention relates to a rotary cylindrical valve, comprising a valve cam; and a valve shaft coupled with the valve cam for rotating the valve cam.

In one embodiment, the rotary cylindrical valve further comprises a valve selection lever affixed to the valve cam for positioning grooves of the valve cam over valve-actuating balls that are secured over microfluidic conduits.

In one embodiment, different angular positions of the valve selection lever permit different combinations of the microfluidic conduits to be opened or closed, based on pressure applied to the valve-actuating balls or cylindrical pins.

In one embodiment, the valve cam comprises a configurable, modular valve cam including different combinations of one-lobe cams and two-lobe cams with their relative angular alignments being set by protrusions that mate with sockets on a face of an adjacent cam.

In one embodiment, the rotary cylindrical valve is actuated by a rotating cylinder that has either detents or protrusions that act upon a ball or a rod or pin to close a desired fluidic channel.

In one embodiment, the rotary cylindrical valve is integrally cast or injection molded as part of a microfluidic system so as to interface with a valve actuator without requiring additional clamping components.

In one embodiment, the valve cam comprises a spindle cam, wherein in operation, valve-actuating ball flow restrictors or actuating pins or rods compress a channel membrane to close microfluidic channels under the actuating elements.

In one embodiment, the spindle cam is adapted to allow activation or deactivation of single or multiple channels in a specific, preprogrammed sequence.

In a further aspect, the invention relates to a rotating cylindrical valve comprising actuators having elevated actuating surfaces for providing pumping functions by sequential compression of a longitudinal fluidic conduit.

In one embodiment, the conduit is either opened or closed depending upon an angular position of the actuators.

In one embodiment, the actuators have a combination of raised and/or recessed regions to enable sequential opening and closing of valves as so to perform different valving and pumping functions.

In one embodiment, the actuators have a variety of widths to control the number of channels being opened or closed.

In one aspect, the invention relates to a valve comprising a valve body being integral to a microfluidic interconnect, the valve body defining a cavity; and a rotary cylindrical valve actuator operably rotating in the cavity.

In one embodiment, within the cavity, there are raised regions beneath which fluidic conduits are located, the raised regions being operably compressed by rotating the rotary cylindrical valve actuator.

In one embodiment, within the cavity, planar surfaces at a top of the cavity maintain an alignment of the rotary cylindrical valve actuator as it rotates and delivers compressive forces to the conduits underneath.

In another aspect, the invention relates to a microfluidic interconnect system comprising a valve body defining a cavity in which a rotary cylindrical valve actuator operates.

In one embodiment, within the cavity, there are raised regions beneath which fluidic conduits are located, the raised regions being operably compressed by regions of the rotary valve actuator without grooves.

In one embodiment, within the cavity, planar surfaces at a top of the cavity maintain an alignment of the rotary cylindrical valve actuator as it rotates and delivers compressive forces to the conduits underneath.

In one embodiment, the conduits connect ports to internal conduits that carry fluids to the connected microfluidic bioreactor, perfusion controller, or other instrument.

In one embodiment, the conduits are formed in either a lower surface of the valve body or an upper surface of a membrane that seals the conduits.

These and other aspects of the invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1A shows a cross-sectional assembly view of a five-layer microfluidic bioreactor according to one embodiment of the invention.

FIG. 1B shows a perspective and other views of a pair of stacked, nesting transwell inserts according to one embodiment of the invention.

FIG. 1C shows schematically a perspective view of multiple interconnecting organ Perfusion Controllers, and a layout of the fluidic connections into, within, and out of a generic organ Perfusion Controller according to one embodiment of the invention.

FIG. 1D shows schematically (left) a diagram of the Run Interconnected Mode showing an interconnect between two separate modules with the interconnecting system valves and fluidic bus lines according to one embodiment of the invention, and (right) a diagram of the Sterilize/Wash Mode showing how the Sterilize/Wash Mode of the interconnect system is achieved by proper setting of the valves on both sides of the interconnect according to one embodiment of the invention.

FIG. 2A shows an exploded view of a two-chamber microfluidic bioreactor according to one embodiment of the invention.

FIG. 2B shows an exploded view of a four-chamber microfluidic bioreactor according to one embodiment of the invention.

FIG. 3A shows an exploded view of a simple threaded clamp housing with two clamp retaining rings (top and bottom) according to one embodiment of the invention.

FIG. 3B shows a perspective view of a two-chamber microfluidic bioreactor using the simple clamp housing according to one embodiment of the invention.

FIG. 3C shows a perspective view of a three-chamber microfluidic bioreactor using the simple clamp housing according to one embodiment of the invention.

FIG. 3D shows a cutaway cross-sectional view of a three-chamber microfluidic bioreactor using the simple clamp housing according to one embodiment of the invention.

FIG. 4A shows an exploded view of a bayonet threaded clamp housing with a clamp retaining ring according to one embodiment of the invention.

FIG. 4B shows an exploded view of a two-chamber bioreactor in which the layers are notched and lower layers have tubing sockets.

FIG. 5A shows an exploded view of a crown threaded clamp housing with multiple clamp retaining rings and fluidic interface supports according to one embodiment of the invention.

FIG. 5B shows a perspective view of a symmetrical microfluidic interface for use with the crown threaded clamp housing according to one embodiment of the invention.

FIG. 5C shows a perspective view of a two-chamber microfluidic bioreactor using the crown clamp housing according to one embodiment of the invention.

FIG. 5D shows a cutaway perspective view of a two-chamber microfluidic bioreactor using the crown clamp housing according to one embodiment of the invention.

FIG. 5E shows a plan layout of six microfluidic bioreactors with symmetric microfluidic interfaces according to one embodiment of the invention, demonstrating how the devices can be multiplexed and positioned within the footprint of a standard six-well plate.

FIG. 5F shows a schematic plan view of an overlapping, two-chamber microfluidic interface for use with the crown threaded clamp housing according to one embodiment of the invention.

FIG. 5G shows a perspective view of a two-chamber microfluidic bioreactor using the overlapping microfluidic interface with a crown clamp housing according to one embodiment of the invention, including the location of optional cut-off valves.

FIG. 5H shows a perspective and end-cross-section detail of a two-chamber microfluidic bioreactor using the overlapping microfluidic interface with a crown clamp housing, detailing the arrangement of patterned microfluidic channels of the two chambers.

FIG. 5I shows a plan and perspective layout of six microfluidic bioreactors using the overlapping microfluidic interface, according to one embodiment of the invention, demonstrating how the devices can be multiplexed and mounted in a caddy that is the size of a six-well plate that in turn is mounted on a carrier with handles.

FIG. 6A shows a cutaway perspective view of a transwell adapter, holding a somatic cell chamber with an attached semipermeable membrane. It is used to culture cells outside of the bioreactor prior to or after incubation in the bioreactor.

FIG. 6B shows a detailed cut away of the interface between a transwell adapter and a somatic cell chamber, illustrating how the somatic cell chamber is held in place.

FIG. 7A shows a schematic cross-section of a symmetrical bioreactor that has been disconnected from external fluidic lines by means of a microfluidic valve actuator assembly, with the valves closed and the ports on both the bioreactor and the external lines are sealed with four removable covers.

FIG. 7B shows a schematic cross-section of a symmetrical bioreactor that is connected to external fluidic lines by means of a microfluidic valve actuator assembly, with the covers removed and the valves open.

FIG. 7C shows a schematic of the operation of the microfluidic valve actuator assembly. The schematic shows Position A (“Run position”), Position B (“Sterilize/Rinse/Dry/Remove Bubbles position”), and Position C (“Disconnect position”).

FIG. 7D shows a grooved rotary cylindrical valve with a ball-capture mechanism according to one embodiment of the invention.

FIG. 7E shows a detailed view of a rotary cylindrical valve cam, and demonstrates how different rotary orientations of the cam correspond to different functional positions of the valve.

FIG. 8 shows a detailed view of a lobed rotary cylindrical valve cam according to one embodiment of the invention.

FIG. 9A shows a schematic of a microfluidic manifold that would be controlled by a rotary cylindrical valve.

FIG. 9B shows a detailed perspective view of a spindle-cam embodiment of a rotary cylindrical valve with housing, engaging valve-actuating balls to control flow in a microfluidic manifold.

FIG. 9C shows a perspective view of a spindle-cam embodiment of a rotary cylindrical valve with housing, engaging cylindrical pins to control flow in a microfluidic manifold.

FIG. 9D shows five images as the spindle cam of the rotary cylindrical valve is turned from one position to another.

FIG. 9E shows cross-sectional and end views of a linear-tumbler rotary cylindrical valve with captured balls.

FIG. 9F shows a side view of a drum-sequencer rotary cylindrical valve with slots that release compressed channels.

FIG. 9G shows a side view of a machined-cam actuator for a rotary cylindrical valve with slots that compress the channels when the cam is rotated over the appropriate channel.

FIG. 10A shows a perspective rendering of a integrally cast valve body for a rotary cylindrical valve

FIG. 10B shows a schematic image an integrally cast valve body for a rotary cylindrical valve, with the four casting pins for the ports and the casting mold for the actuator region still in place.

FIG. 10C shows the upper surface of the mold for the integrally cast rotary cylindrical valve.

FIG. 10D shows the lower surface of the mold for the integrally cast rotary cylindrical valve.

FIG. 10E shows the cylindrical actuator with compression-release grooves for the integrally cast rotary cylindrical valve.

FIG. 10F shows an end view of a cylindrical actuator in the actuator region of the integrally cast valve body.

FIG. 10G shows a normally-open rotating cylindrical valve with elevated actuating surfaces rather than recessed ones.

FIG. 10H shows how a rotating cylindrical valve with elevated actuating surfaces can also provide pumping functions.

FIG. 11A shows a layout of a 3-element bioreactor chain according to one embodiment of the invention.

FIG. 11B shows a perspective view of three symmetrical crown-clamp housings with serially connected bioreactors.

FIG. 11C shows a plan schematic of three symmetrical crown-clamp housings, each of which contains a spiral-channel membrane bioreactor.

### DETAILED DESCRIPTION OF THE INVENTION

The invention will now be described more fully hereinafter with reference to the accompanying drawings, in which exemplary embodiments of the invention are shown. The invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the invention or of any exemplified term. Likewise, the invention is not limited to various embodiments given in this specification.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the invention.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. As used herein, the term “and/or” includes any and all combinations of one or more of the associated listed items.

The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

Our U.S. patent application Ser. No. 15/776,524 (the '524 application), filed May 16, 2018, entitled “Multicomponent Layered and Stackable Microfluidic Bioreactors and Applications of Same”, by John P. Wikswo et al., which is incorporated herein by reference in its entirety, discloses a device that specifically meets unaddressed needs in the art: a device that provides shear-flow induced polarization of endothelial cells; a device that presents small, physiologically realistic fluid volumes to cells that do not dilute concentration-based signaling molecule feedback systems to the point of loss of function or detectability; a modular, stackable device that allows the inclusion of more than two cell types, and the ability to use electrical recordings to monitor neural cell activity in situ.

Our another U.S. patent application Ser. No. 16/012,900 (the '900 application), filed Jun. 20, 2018, entitled “Interconnections of Multiple Perfused Engineered Tissue Constructs and Microbioreactors, Multi-microformulators and Applications of Same”, by John P. Wikswo et al., which is incorporated herein by reference in its entirety, discloses a device that specifically meets unaddressed needs in the art: a device that interconnects separate microfluidic modules in a sterile, low volume manner; a device that avoids fluid loss and introduction of air bubbles; a device that allows ready sterilization of the interior and exterior of the fluid interconnect pathways; a device that allows timed variation in the concentration of key drugs, nutrients, or toxins over an extended period of time. The need for these capabilities is reviewed by Wikswo et al, [3]. The need for proper scaling of the microfluidic bioreactor and tubing volumes to the cell volumes is discussed in Wikswo et al. [4]. Neither article presents specific solutions to these needs.

Our previous invention of the normally closed rotary planar valve [5] presents a valve concept that can be readily implemented in microfluidics, and our '900 application presents a toggle valve suitable for operating a simple means to sterilize, wash, and connect two bioreactor modules. Neither of these inventions is optimized for the sterilization, wash, and connect operations that are needed in the invention described in the current patent application.

Objectives of this invention are, among other things, to refine, extend and integrate the devices disclosed in our '524 application and '900 application.

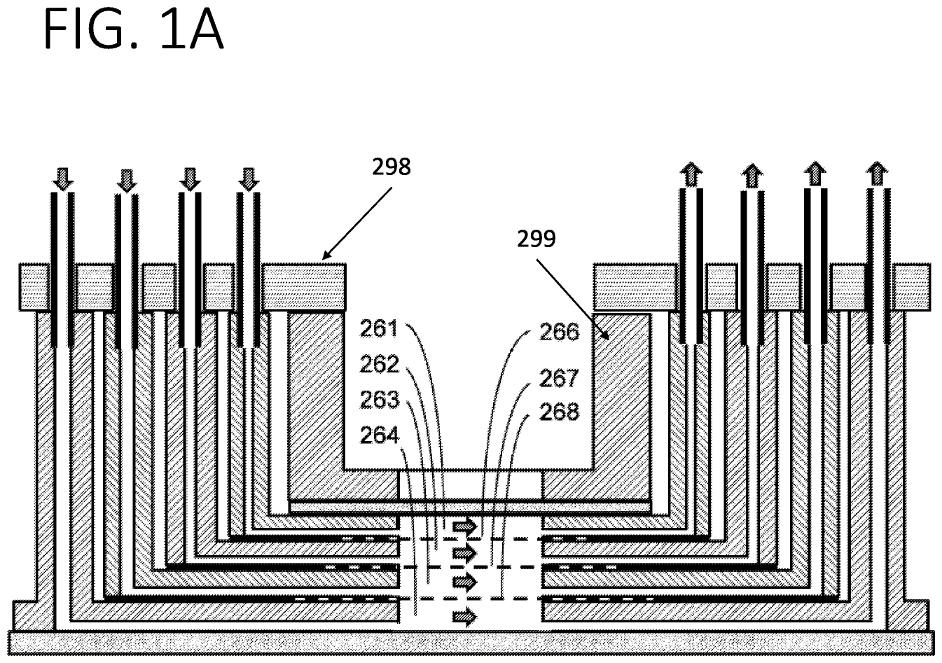

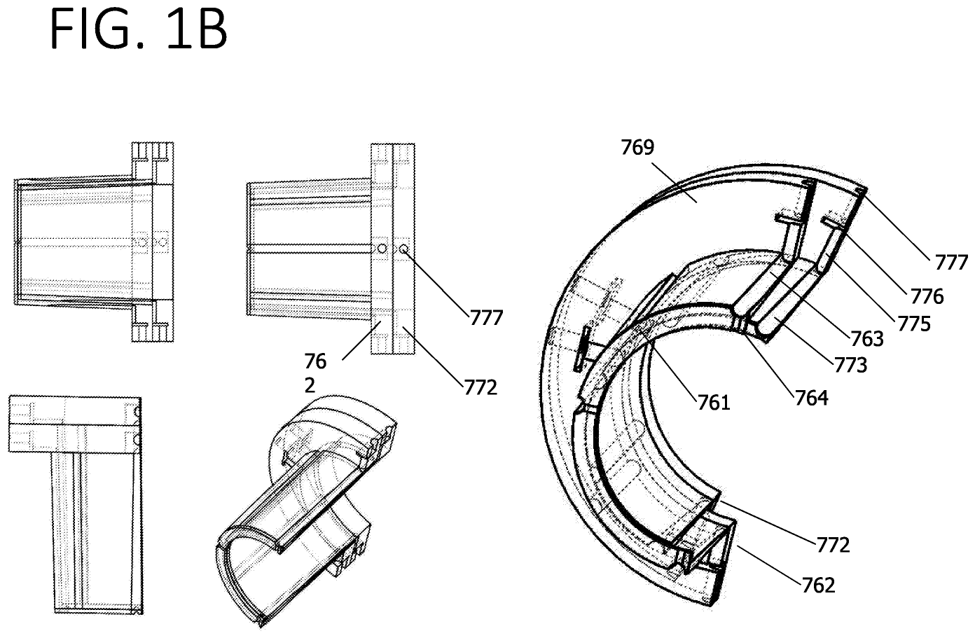

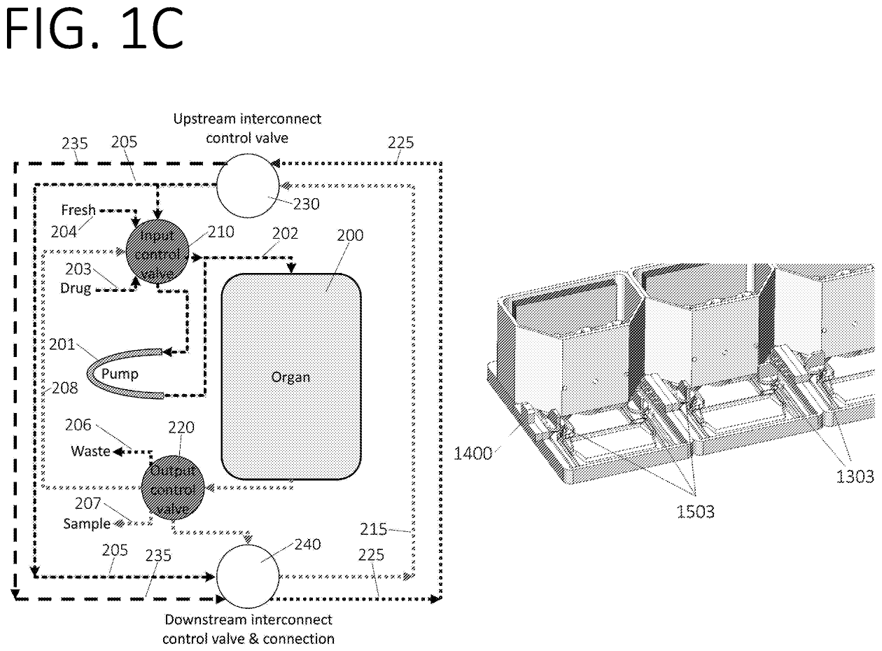

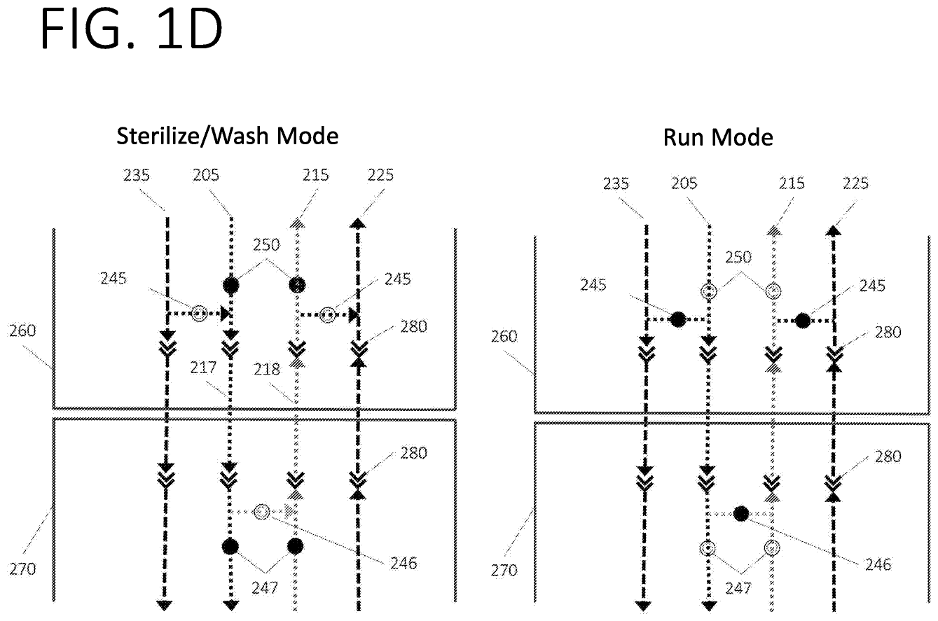

In certain aspects of the invention, the newly invented device incorporates elements disclosed in the '524 application and the '900 application, and primarily addresses shortcomings that previously made the two earlier technologies difficult to combine into a functional whole. The device does not incorporate all the functions of the two earlier devices, but embodies the functions from each that are most valuable when combined and integrated in a manner that was not obvious at the time of the '524 application and the '900 application. In some embodiments, the device also provides an improvement to a function of one of the devices disclosed in the '524 application and the '900 application. FIGS. 1A-1D summarize the elements of the two earlier technologies that have been combined and improved in the invention.

FIG. 1A shows a layered, openable microfluidic bioreactor in cross-section. The device includes nested bioreactor layers with embedded conduits and bottom openings covered with semi-permeable membranes (266-268). The shallow spaces between the membranes and the outer perimeter of each chamber provide a volume where cells can be grown (261-264). This volume can be of any shape, and as presented in the '524 application, the outermost profile of each layers is rectangular, with the embodiment shown indicating that the outer portions of each layer provide space for using vertical tubing connections to access the fluidics in that layer, and each layer fits within the space provided for it by the next layer outwards. The limitation of this type of layered bioreactors, addressed by the invention, is that the ability of the clamp mechanism (298 and 299) to seal all of the layers is determined by how well the thickness of each layer can be controlled such that there is uniform sealing pressure applied between each layer, which may complicate the fabrication of the individual components and increase the cost of manufacture.

FIG. 1B shows an alternative, cylindrically symmetric stackable bioreactor system that more closely resembles a classic transwell insert. The drawing illustrated two of the nested inserts in cutaway for detail. The figure shows two inner inserts 762 and 772, with the insert 772 inside of the insert 762. The vertical channels shown in FIG. 1B are 763 and 764. In addition, there are short radial channels, such as the radial channel 764, which allow flow down the vertical channel 763 to be able to enter the chamber above the filter that would be bonded to the lowermost surface 761 of the insert 762. The insert 772 has a top flange 779 and the insert 762 has a top flange 769. The thickness of these flanges must be sufficient to allow the connection of tubing to the drilled or molded port 777 that connects to the inner channel 773 by means of an upper radial channel 775. To simplify making the fluidic connection between the flange 779 and the upper radial channel 775, a notch 776 is molded into the lower surface of the flange 779 to allow the port to be in the middle of the thickness of the flange while the channel 775 can be on the lower surface of the flange 779.

This system of nested cup-like inserts is limited in several ways. First, with the current design, the addition of layers changes the footprint of the system as a whole. This makes standardization more difficult, and interface of the stacked insert bioreactor with other components, like interconnect valves, more complicated. Because the size difference between inner and outer cup defines the number of possible layers in a given configuration, more pair combinations are necessary to ensure flexibility in device application, and more parts are potentially left unused. Second, because integrity of the mated surfaces of the nested inserts is critical for proper sealing and operation of the device, the magnitude and uniformity of the clamping force required to hold the nested inserts together is also critical. However, no clamp is provided as part of the device, and one must assume some unspecified means to hold the stack together. Finally, the cup-like shape of the inserts present a large, multi-surface area that must remain sealed for proper operation, complicating the design and fabrication of the injection molds to create each of the different-sized nested layers. In addition, this design does not ensure that the vertical clamping that holds the inserts together will be uniformly applied to the tangential surfaces that must be sealed to prevent leaks between the connections of the rim of each insert and the chambers created at the bottom by the vertical space between two adjacent inserts.

FIG. 1C shows embodiments of the means to connect multiple organ-on-chip modules, such as bioreactors with self-contained perfusion controllers, microclinical analyzers, heart-lung systems, or other associated modules, disclosed in the '900 application. The principal subject of the invention disclosed in the '900 application is to provide a means to sterilize, wash, eliminate bubbles and break fluidic connections into, within, and out of a generic organ Perfusion Controller (PC). One embodiment of the invention is shown (left) as well as a perspective view of multiple interconnecting organ Perfusion Controllers (right). As shown in the left layout, a generic organ 200 is directly driven by a local Perfusion Controller pump 201 via an input feed line 202. Input to this pump is selected by an input control valve 210 which can select either drug 203, fresh media 204 or input from the arterial bus 205, for example. Output from the organ 200, controllable via the output control valve 220, can either be delivered to waste 206, collected as a sample for further analysis 207, recirculated 208 to the input control valve, or sent to the venous bus 215, for example. The organ chip has the ability to address the arterial bus line 205 and the venous bus line 215. Different topologies of valves could implement these and other functions for localized control of each organ. Input from the arterial bus line 205 can be selected by the upstream interconnection bus valve 230 and routed by the input control valve 210 to be delivered to the organ chip 200. Similarly, output from the organ 200 can be selected by the output control valve 220 and routed by the downstream interconnection bus control valve 240 to the venous bus line 215. Note that flow of the arterial and venous lines is clockwise in this possible realization of the interconnectable Perfusion Controller. The bottom perspective view shows multiple interconnecting organ Perfusion Controllers. Fluidic interface between the organs is controlled by interconnection bus control valve knobs 1303. An interconnection cover is required to complete the fluidic circuit. Note that in the leftmost module three control knobs 1503 are set to the Sterilize/Wash Mode to sterilize the interconnects of the leftmost module, whereas upstream control knobs 1303 are set in the Run position.

FIG. 1D shows the details of the Sterlize/Wash mode (left) and the Run mode (right) of the interconnection valves disclosed in the '900 application. The left diagram is a schematic illustration of the Sterilize/Wash Mode showing the Sterilize/Wash Mode of the interconnect system is achieved bewteen two separate integrated organ microfluidics (IOM) modules 260 and 270, whether they are Perfusion Controllers, MicroClincal Analyzers, MicroFormulators, Cardiopulmonary Assist Modules, or other devices, with the interconnecting system valves and fluidic bus lines. In the Sterilize/Wash Mode, valves blocking the horizontal connections 245 and 246 are now open, allowing flow of wash/sterilize/rinse solution 235 through the arterial 217 and venous 218 interconnect lines, which are now isolated from the corresponding lines in the body of the module by closed valves 247 and 250. Note that there is an asymmetry between the valves at the bottom (downstream) side of the IOM 260 and the top (upstream) side of the IOM 270. The valves 245 in the upper IOM 260 engage and disengage connections between wash and arterial, and venous and waste, in the fluidics within the upper IOM 260. In the lower IOM 270, there is simply a valved connection 246 between the arterial and venous interconnect lines to enable the sterilization and wash fluids to pass through all four interconnect lines, thereby sterilizing and washing the entire interconnect, while valves 247 to shut off the arterial and venous lines so that sterilize/wash fluid cannot enter the organ fluidics in the lower IOM 270. Note that there may be short stubs of fluid immediately adjacent to a closed control point that are not washed by through-flow, but the short length of these stubs and the convective and diffusive movement of sterilizing and washing solutions during the sterilization and wash steps will render these stubs both sterile and then washed. The right diagram is a schematic illustration of the Run mode showing an interconnect between two separate IOM modules 260 and 270 with the interconnecting system valves and fluidic bus lines. There are four parallel lines that cross between the upper proximal module 260 and lower distal module 270: a wash line 235 for delivery of a detergent, sterilant, and rinse solutions; an arterial line 205 to deliver fresh media; a venous line 215 to return conditioned media to the Cardiopulmonary Assist Module; and a waste line 225 into which the detergent, sterilant, and rinse solutions are removed from the system. Solid circles are closed valves, and open circles are open valves. The arrowheads designate that fluid is flowing in that direction. Line segments without fluid flow are without arrowheads. Hence this system provides fluidic connection for wash/sterilize/rinse 235 and arterial 205 solutions to flow from the proximal module 260 to the distal module 270, and venous 215 and waste 225 solutions to flow from the distal module 270 to the proximal module 260. The double-headed arrows 280 represent the interruptible connection between modules 260 and 270. These lines are controlled by open (open circles) control valve points 250 and 247 and closed (solid circles) control valve points 245 and 246 to route fluid either independently along the parallel lines in the Run Interconnected Mode (right diagram), or to wash the interconnects in the SterilizeWash Mode (left diagram), or the Run Isolated Mode.

The embodiments present in the '900 application are limited to the control of microbioreactors integral to a Perfusion Controller module and management of interconnections between modules. Nothing is presented either about how separate chambers within a microfluidic bioreactor can be sealed together or connected or disconnected from their associate perfusion controller pumps and valves.

In one aspect, the invention relates to a multichamber bioreactor comprising multiple planar layers stacked on each other defining at least one chamber, and secured by a clamping mechanism.

In one embodiment, each chamber is implemented from a separate fluidic layer, with each fluidic layer having ports and valves independent of the other layers.

In one embodiment, the clamping mechanism includes a housing and retaining means received in the housing and configured to generate a controlled and uniform pressure to secure the stacked multiple planar layers in the housing.

In one embodiment, the stacked multiple planar layers comprise an endothelial microfluidic disc; at least one somatic cell chamber layer disposed on the endothelial microfluidic disc; a microfluidic perfusion disc disposed on the at least one somatic cell chamber layer; a pressure plate disposed on the at least one somatic cell chamber layer; and at least one membrane, each member disposed between the endothelial microfluidic disc and the at least one somatic cell chamber layer, and between two adjacent somatic cell chamber layers when the at least one somatic cell chamber layer has two or more somatic cell chamber layers. The stacked multiple planar layers are placed on a base plate, received in the housing.

In one embodiment, the stacked multiple planar layers comprise microfluidic perfusion discs disposed on each side of the at least one somatic cell chamber layer; a pressure plate disposed on the at least one somatic cell chamber layer; and the at least one membrane, each member disposed between the endothelial microfluidic disc and the at least one somatic cell chamber layer, and between two adjacent somatic cell chamber layers when at the least one somatic cell chamber has two or more somatic cell chamber layers. The stacked multiple planar layers are placed on a base plate, received in the housing. The exact topology and height of the perfusion channels in the microfluidic perfusion or endothelial disks can be adjusted as required by the specific cells and tissues to be cultured in the bioreactor.

In one embodiment, each of the pressure plate and the microfluidic perfusion disc has a plurality of through holes defined therein, and aligned to each other in the stacked multiple planar layers, such that a plurality of tubes with flexible lengths is insertable into the through holes of the pressure plate and the microfluidic perfusion disc for connecting to an individual layer.

In one embodiment, the microfluidic perfusion disc has notches formed on its edge and tubing sockets protruded from the microfluidic perfusion disc for connecting a plurality of tubes to an individual layer; the somatic cell chamber layer has tubing sockets protruded from the somatic cell chamber layer for connecting the plurality of tubes to an individual layer; and the pressure plate has notches formed its edge, such that, as assembled, the tubing sockets of the somatic cell chamber layer are received in the tubing sockets of the microfluidic perfusion disc, which are in turn received in the notches of the pressure plate, so as to allow each upper layer to be inserted over a lower layer without having to thread the plurality of tubes through individual holes in each upper layer.

In one embodiment, the multichamber bioreactor further includes a transwell adapter for accommodating the at least one somatic cell chamber layer to allow culture of cells independent of the bioreactor prior to insertion of, or after extraction of the at least one somatic cell chamber layer.

In one embodiment, the housing has a threaded inner surface, wherein the retaining means includes at least one threaded retaining ring being operably threaded into the housing to secure the stacked multiple planar layers in the housing.

In one embodiment, the bioreactor housing has a removable lower retaining ring rather than a retaining lip that is part of the housing. In either case, a separate retaining ring to compress the bioreactor layers.

In one embodiment, the housing has slots formed in a wall of the housing, and wherein the retaining means includes a retaining ring having pins radially protruded from a peripheral side of the retaining ring being operably fitted into the slots of the housing 401 to secure the stacked multiple planar layers in the housing. In one embodiment, the slots are L-shape slots.

In one embodiment, the retaining means includes an expanding clamp pressing outwards against an inner surface of the housing to be held in place by force of friction between the sides of the expanding clamp and the inner surface of the housing.

In one embodiment, the housing includes an internally and externally threaded, notched crown housing, and wherein the retaining means includes an externally-threaded retaining ring;

two internally threaded retaining rings; a fluidic interface bottom support; a fluidic interface top support; and a microfluidic interface disposed between the fluidic interface bottom support and the fluidic interface top support, wherein the fluidic interface bottom and top supports are designed to provide mechanical support for insertion of tubes or ribbon connectors into fluidic and to protect the fluidic during handling of the multichamber bioreactor.

In one embodiment, the microfluidic interface includes conduits embossed on underside and vertical ports. In one embodiment, the two ports and channels furthest from an axis of the conduits are connected to an endothelial chamber of the endothelial microfluidic disc, and the four ports and channels closest to the axis are connected to a stromal cell chamber of the somatic cell chamber layer. In one embodiment, the ports and channels for one layer are directed towards one slot of the crown housing, and the ports for the other layer are directed to a different slot of the crown housing.

In one embodiment, the microfluidic interface further includes shut-off valves coupled between the conduits and the ports.

In one embodiment, the multichamber bioreactor is operably connected to a perfusion controller, and/or a second, downstream multichamber bioreactor, by at least one connector assembly.

In one embodiment, the multichamber bioreactor further includes valves integrated into input and output fluidic lines to enable sterilization and washing of open ports of the fluidic connections and elimination of bubbles during the connection process before the ports are connected to the bioreactor chambers and external support equipment, and eliminate leakage of fluid when disconnected.

In one embodiment, the multichamber bioreactor is insertable into a holder whose size is consistent with a standard well plate and whose individual chambers can be cultured separately prior to assembly, with a rotating or friction-fit retaining ring or rings within a structure with a hollow cylindrical region that aligns and holds components together without need for irreversible bonding or multiple fasteners or levers.

In one embodiment, the multichamber bioreactor is capable of being rapidly dis-assembled for analysis of cellular contents or for their interfacing to other systems and components.

In one embodiment, the multichamber bioreactor is operable with an arbitrary number of chambers in a vertical stack without modifications to hardware.

In yet another aspect, the invention relates to a rotary cylindrical valve, comprising a valve cam; and a valve shaft coupled with the valve cam for rotating the valve cam.

In one embodiment, the rotary cylindrical valve further includes a valve selection lever affixed to the valve cam for positioning grooves of the valve cam over valve-actuating balls that are secured over microfluidic conduits.

In one embodiment, different angular positions of the valve selection lever permit different combinations of the microfluidic conduits to be opened or closed, based on pressure applied to the valve-actuating balls or cylindrical pins.

In one embodiment, the valve cam includes a configurable, modular valve cam including different combinations of one-lobe cams and two-lobe cams with their relative angular alignments being set by cell chamber layer 283 with sockets on a face of an adjacent cam.

In one embodiment, the rotary cylindrical valve is actuated by a rotating cylinder that has either detents or protrusions that act upon a ball or a rod or pin to close a desired fluidic channel.

In one embodiment, the rotary cylindrical valve is integrally cast or injection molded as part of a microfluidic system so as to interface with a valve actuator without requiring additional clamping components.

In one embodiment, the valve cam includes a spindle cam, wherein in operation, valve-actuating ball flow restrictors or actuating pins or rods compress a channel membrane to close microfluidic channels under the valve-actuating balls.

In one embodiment, the spindle cam is adapted to allow activation or deactivation of single or multiple channels in a specific, preprogrammed sequence.

In a further aspect, the invention relates to a rotating cylindrical valve comprising actuators having elevated actuating surfaces for providing pumping functions by sequential compression of a longitudinal fluidic conduit.

In one embodiment, the conduit is either opened or closed depending upon an angular position of the actuators.

In one embodiment, the actuators have a combination of raised and/or recessed regions to enable sequential opening and closing of valves as so to perform different valving and pumping functions.

In one embodiment, the actuators have a variety of widths to control the number of channels being opened or closed.

In one aspect, the invention relates to a valve comprising a valve body being integral to a microfluidic interconnect, the valve body defining a cavity; and a rotary cylindrical valve actuator operably rotating in the cavity.

In one embodiment, within the cavity, there are raised regions beneath which fluidic conduits are located, the raised regions being operably compressed by rotating the rotary cylindrical valve actuator.

In one embodiment, within the cavity, planar surfaces at a top of the cavity maintain an alignment of the rotary cylindrical valve actuator as it rotates and delivers compressive forces to the conduits underneath.

In another aspect, the invention relates to a microfluidic interconnect system comprising a valve body defining a cavity in which a rotary cylindrical valve actuator operates.

In one embodiment, within the cavity, there are raised regions beneath which fluidic conduits are located, the raised regions being operably compressed by regions of the rotary valve actuator without grooves.

In one embodiment, within the cavity, planar surfaces at a top of the cavity maintain an alignment of the rotary cylindrical valve actuator as it rotates and delivers compressive forces to the conduits underneath.

In one embodiment, the conduits connect ports to internal conduits that carry fluids to the connected microfluidic bioreactor, perfusion controller, or other instrument.

In one embodiment, the conduits are formed in either a lower surface of the valve body or an upper surface of a membrane that seals the conduits.

Without intent to limit the scope of the invention, exemplary embodiments of the invention are given below. For purposes of clarity, the same reference numbers will be used in the drawings to identify similar elements.

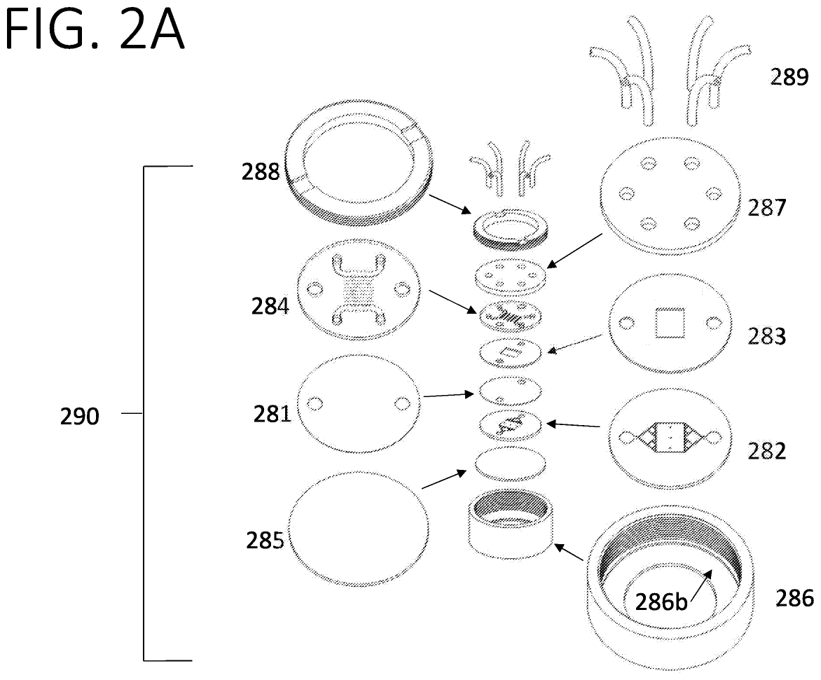

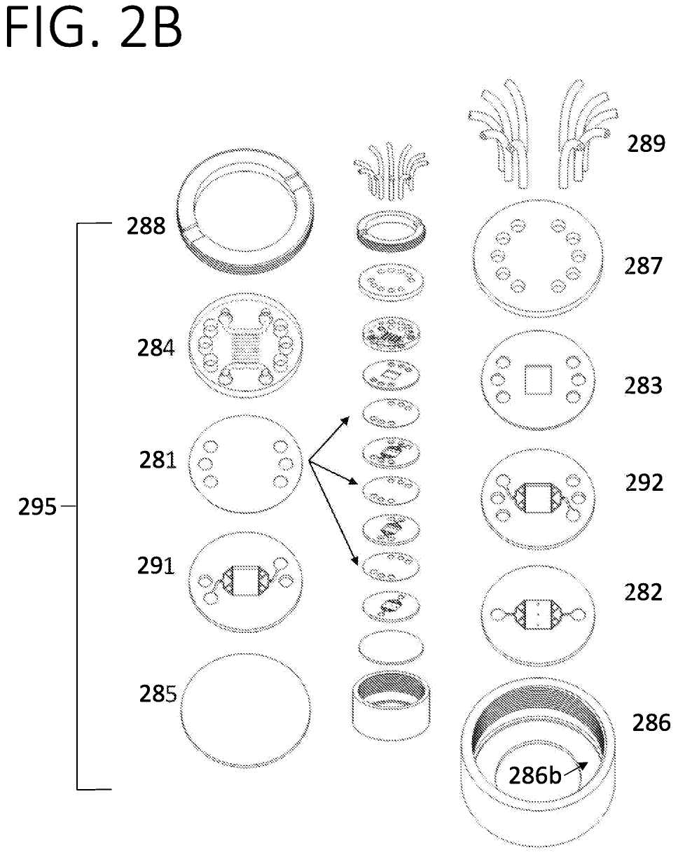

Referring to FIGS. 2A-2B, a layered, planar multi-chamber microfluidic bioreactor according to certain embodiments is shown. Of them, FIG. 2A explains a concept by which a layered, planar, multi-chamber bioreactor can be fabricated and assembled.

Referring to FIG. 2A, the two-chambered assembly of a microfluidic bioreactor 290 includes a microfluidic device that has features that can be created by compressing individual components, and which can be fabricated by casting, embossing, injection molding, or machining, to form an integral unit. Membrane 281 is layered between an endothelial microfluidic disc 282 and a somatic cell chamber layer 283. A microfluidic perfusion disc 284 is mated to the top of the somatic cell chamber 283 to provide efficient nutrient delivery and waste removal to the somatic cells. This microfluidic assembly is then placed on a glass base 285 which fits inside the bioreactor housing 286 and rests on the bottom retaining lip 286b. A rigid pressure plate 287 is then aligned to the perfusion disc 284 and the whole assembly is compressed by an upper, externally threaded retaining ring 288 which is threaded into the internal threads of the bioreactor housing. Flexible lengths of tubing 289 are then threaded through the holes in the pressure plate 287, and inserted into the holes in the perfusion disc 284.

Referring to FIG. 2B, an exploded view of a four-chambered assembly of a microfluidic bioreactor 295, shows a microfluidic device that has features that can be created by compressing individual components, which can be fabricated by casting, embossing, injection molding, or machining, to form an integral unit. Membranes 281 are layered between an endothelial microfluidic disc 282 and a somatic cell chamber layer 291, between the somatic cell chamber layer 291 and a somatic cell chamber layer 292, and between the somatic cell chamber layer 292 and a somatic cell chamber layer 283. A microfluidic perfusion disc 284 is mated to the top of the somatic cell chamber layer 283 to provide efficient nutrient delivery and waste removal to the somatic cells. This microfluidic assembly is then placed on a glass base 285 which fits inside the bioreactor housing 286 and rests on the bottom retaining lip 286b. A rigid pressure plate 287 is then aligned to the perfusion disc and the whole assembly is compressed by a top, externally threaded retaining ring 288 which is threaded into the internally threaded bioreactor housing. Flexible lengths of tubing 289 are then passed through the holes in the pressure plate 287, and inserted into the holes in the perfusion disc 284.

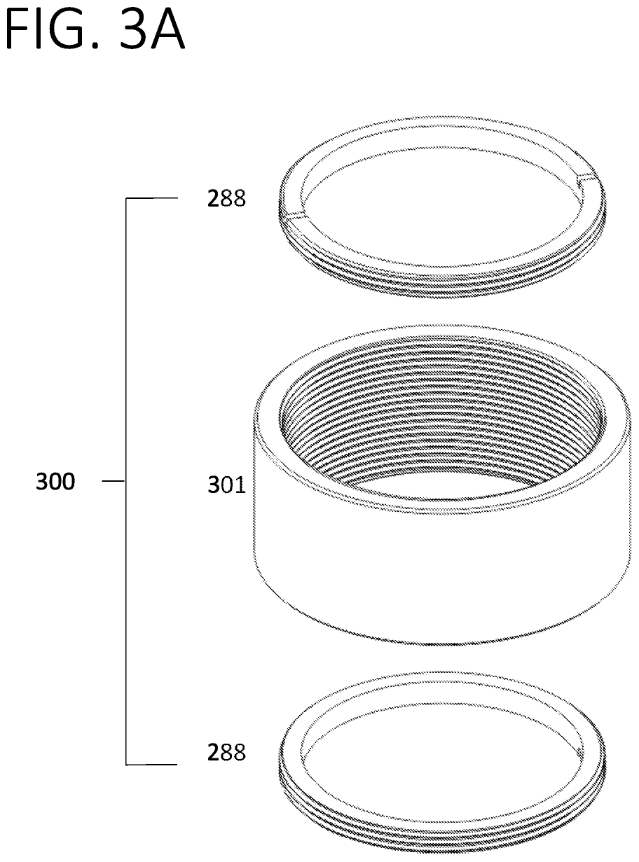

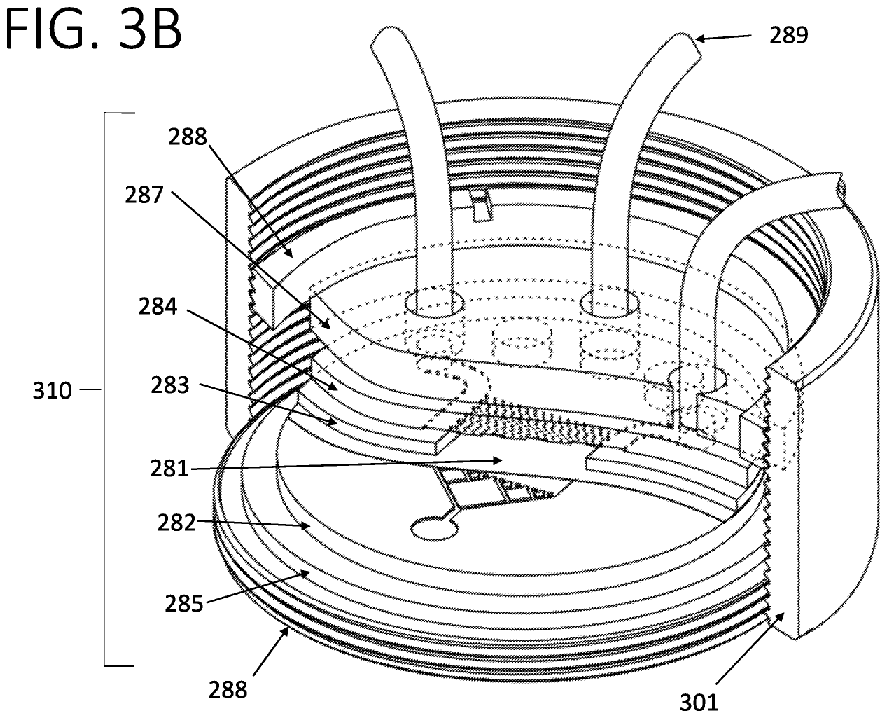

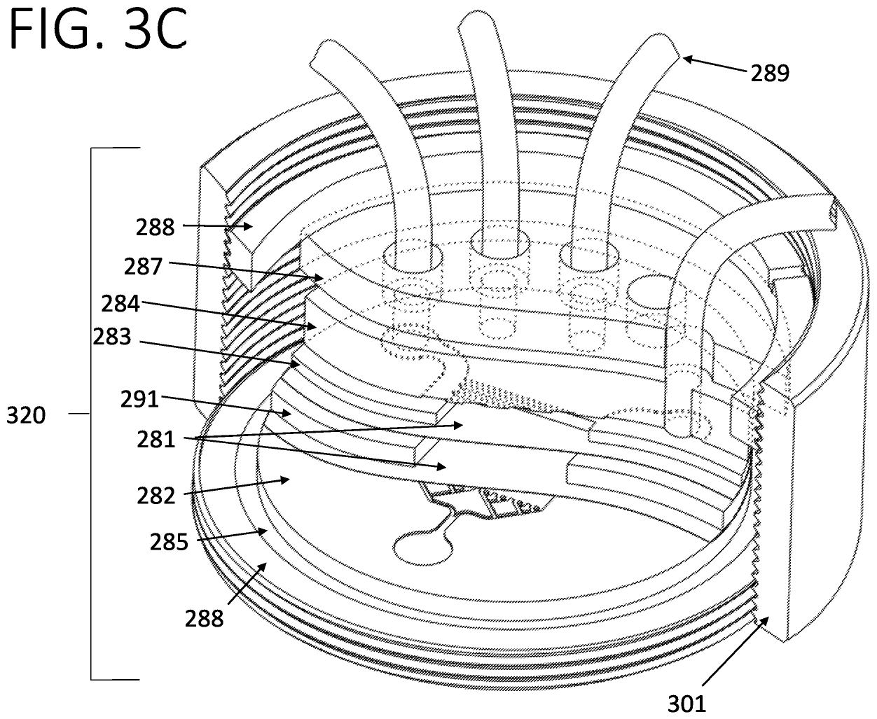

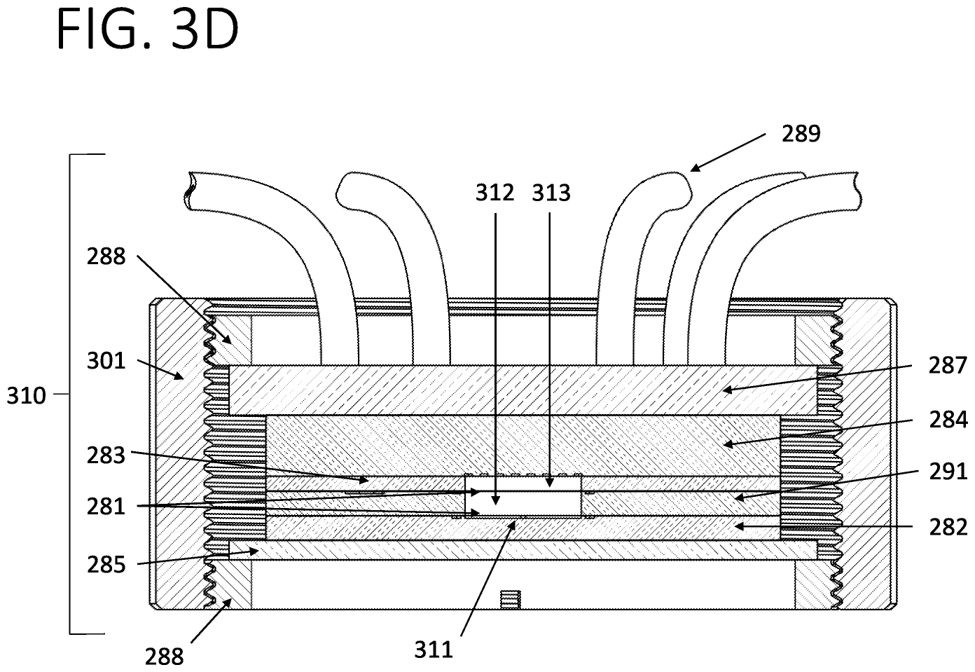

Referring to FIG. 3A, an exploded view of a clamping device 300 is shown according to one embodiment of the invention. In the exemplary embodiment, the clamping device 300 includes an internally threaded housing 301, and two externally-threaded retaining rings 288, that are used to generate a controlled and uniform pressure to clamp a layered, planar multi-chamber microfluidic bioreactor according to certain embodiments of the invention. The use of the bottom retaining lip 286b in FIG. 2 provides the same function as the lower retaining ring 288, and may be less expensive to fabricate and thinner than the lower ring 288.

Referring to FIG. 3B, the isometric cutaway perspective view of a two-chambered assembly of a microfluidic bioreactor 310 demonstrates that it includes elements of a microfluidic bioreactor 290 (shown in FIG. 2A) with the clamping device 300. Bioreactor 310 includes a microfluidic device that has features that can be created by compressing individual components, which can be fabricated by casting, embossing, injection molding, or machining, to form an integral unit. Membrane 281 is layered between an endothelial microfluidic disc 282 and a somatic cell chamber layer 283. A microfluidic perfusion disc 284 is mated to the top of the somatic cell chamber layer 283 to provide efficient nutrient delivery and waste removal to the somatic cells. This microfluidic assembly is then placed on a glass base 285 which fits inside the bioreactor housing 301 and rests on a bottom threaded retaining ring 288. A rigid pressure plate 287 is then aligned to the perfusion disc and the whole assembly is compressed by a top threaded retaining ring 288 which is threaded into the bioreactor housing. Flexible lengths of tubing 289 are then threaded through the holes in the pressure plate, and inserted into the holes in the perfusion disc.

Referring to FIG. 3C, an isometric cutaway view of a three-chambered assembly of a microfluidic bioreactor 320 reveals that this embodiment has elements of the assembly 295 shown in FIG. 2B, with the clamping device 300 shown in FIG. 3A. Bioreactor 320 includes a microfluidic device that has features that can be created by compressing individual components, which can be fabricated by casting, embossing, injection molding, or machining, to form an integral unit. Membranes 281 are layered between an endothelial microfluidic disc 282, and a somatic cell chamber layer 291, and between the somatic cell chamber layer 291 and a somatic cell chamber layer 283. A microfluidic perfusion disc 284 is mated to the top of the somatic cell chamber layer 283 to provide efficient nutrient delivery and waste removal to the somatic cells. This microfluidic assembly is then placed on a glass base 285 which fits inside the bioreactor housing 301 and rests on a bottom threaded retaining ring 288. A rigid pressure plate 287 is then aligned to the perfusion disc and the whole assembly is compressed by a top threaded retaining ring 288 which is threaded into the bioreactor housing. Flexible lengths of tubing 289 are then threaded through the holes in the pressure plate 287, and inserted into the holes in the perfusion disc 284.

FIG. 3D is a cross section cutaway view of a three-chambered assembly of a microfluidic bioreactor 320 comprised of elements of the assembly 295 shown in FIG. 2B, with the clamping device 300 showing three cell layers. The three cell layers are formed as illustrated in the cutaway view. The first cell layer is the endothelial cell layer 311, formed between the membrane 281 and the endothelial microfluidic disc 282. The second cell layer is a somatic cell layer 312, formed in the chamber in the somatic cell chamber layer 291, between the membranes 281 and the endothelial cell layer 311. The third cell layer is a somatic cell layer 313, formed in the chamber defined by the cell chamber layer 283 between the membrane 281 and the microfluidic perfusion disc 284.

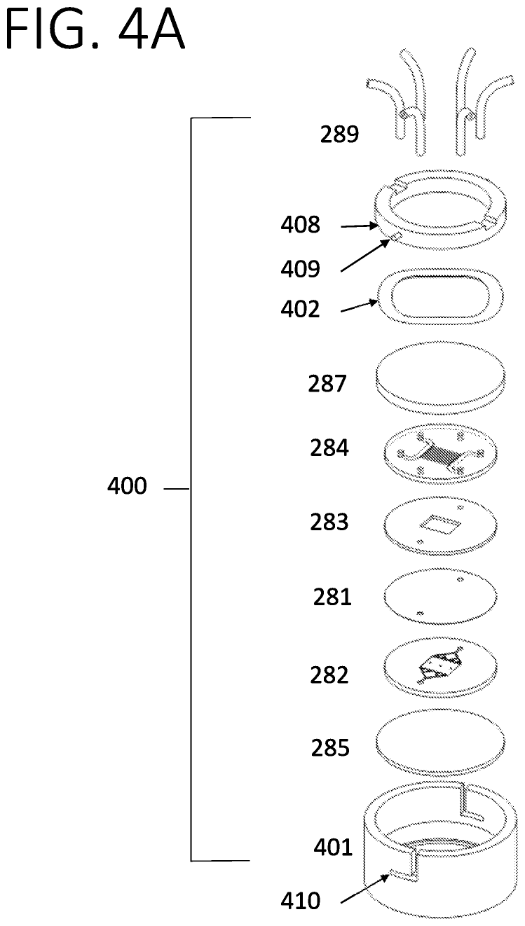

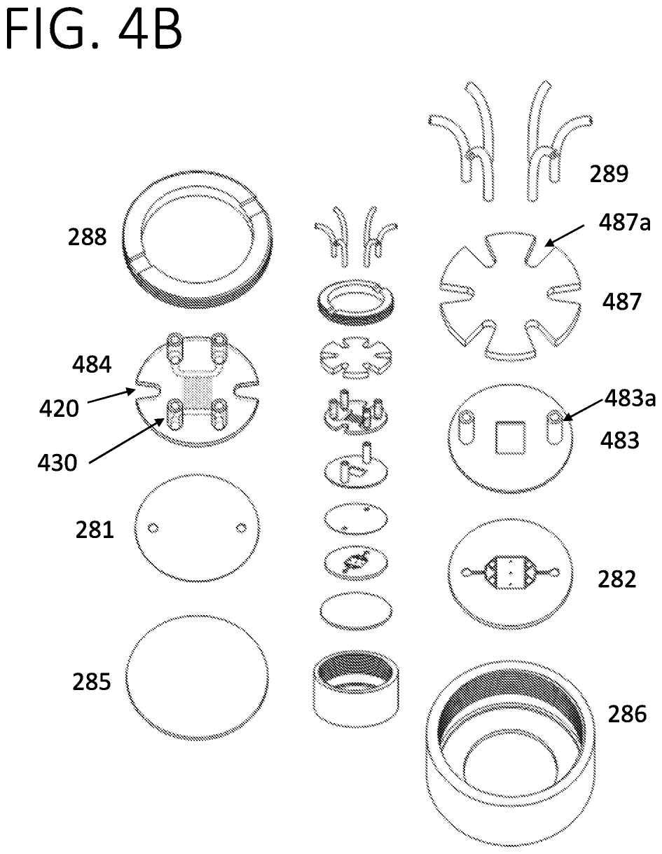

FIG. 4A shows an exploded view an embodiment that utilizes a bayonet clamping device 400 composed of a slotted housing 401 with a bottom retaining lip, leaf spring washer 402, and a pinned retaining ring 408. Pins 409 on the retaining ring 408 fit into slots 410 on the slotted housing 401. As shown, the slots are cut through the full thickness of the cylindrical wall of the housing, but in another embodiment, the slots could be cut only into the inner cylindrical surface of the housing to make the housing wall stronger. Elements of the above described assembly 295 can be clamped using the bayonet clamp 400 in the same manner as with the clamp 300. In this embodiment, the leaf spring washer 402 is compressed between the pinned retaining ring 408 and the rigid pressure plate 287. In another embodiment, the pins 409 would not be necessary were an expanding clamp pressing outwards against the inner wall of the housing 401 to be held in place by the force of friction between the sides of the expanding clamp and the inner surface of the housing wall. The primary advantages of these two embodiments are that the bioreactor can be quickly disassembled in order to extract cells for metabolomic, proteomic, phosphoproteomic, lipidomic, epigenomic, or transcriptomic analysis, and that these types of clamping strategies are readily applicable to automated operation with a robot.

All embodiments up to this point have utilized discrete tubes to access the fluidic networks and chambers in each layer. Since this approach requires that the tubes connected to lower layers need to pass down through the upper layers, it is not possible to combine layers that have already been intubated. The embodiment shown in FIG. 4B shows, among other things, the microfluidic perfusion disc 484 has notches 420 formed thereon to allow each upper layer to be inserted over a lower layer without having to thread the tubes through individual holes in each upper layer, and tubing sockets (posts) 430 that provide a means to connect tubing 289 to an individual layer. The somatic cell chamber 483 also has tubing sockets (posts) 483a for connecting tubing 289 to an individual layer. In addition, the rigid pressure plate 487 includes notches 487a formed thereon for accommodating the tubing sockets 430 and the tubing sockets 483a so as to allow each upper layer to be inserted over a lower layer without having to thread the tubes through individual holes in each upper layer.

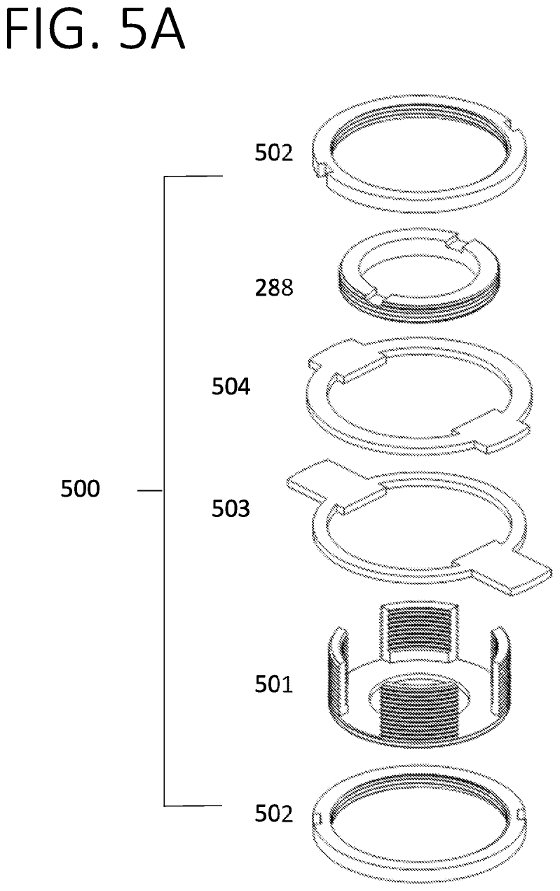

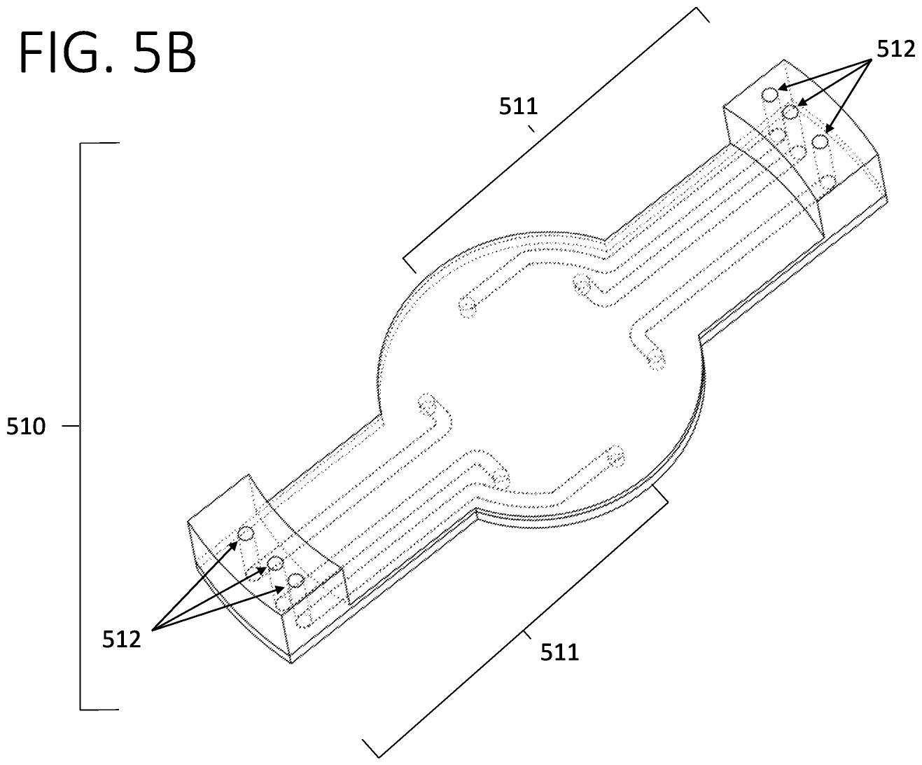

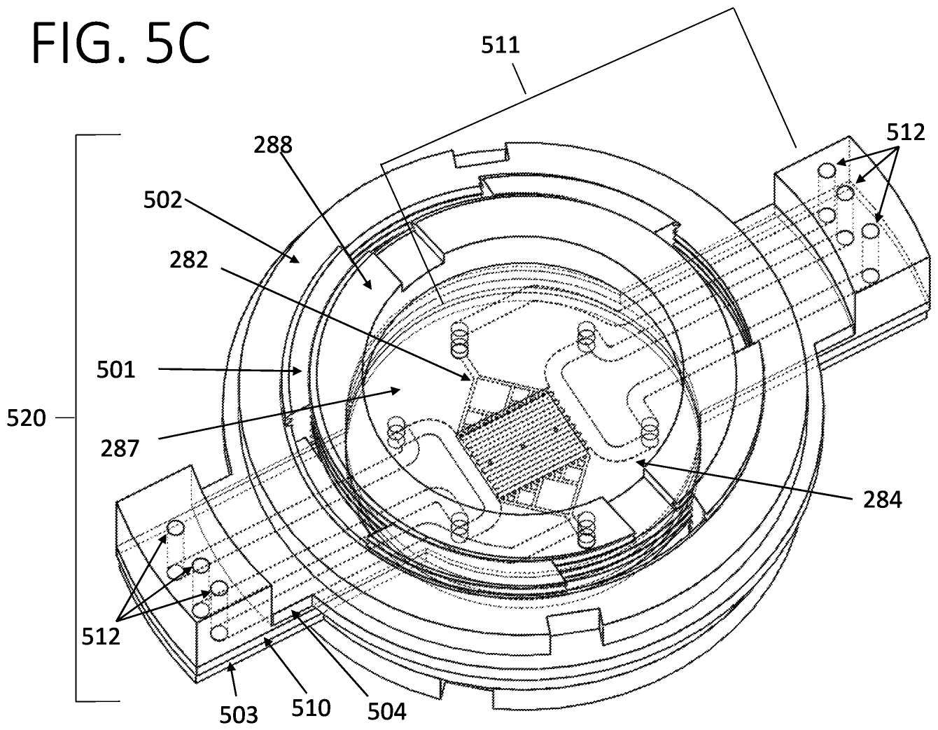

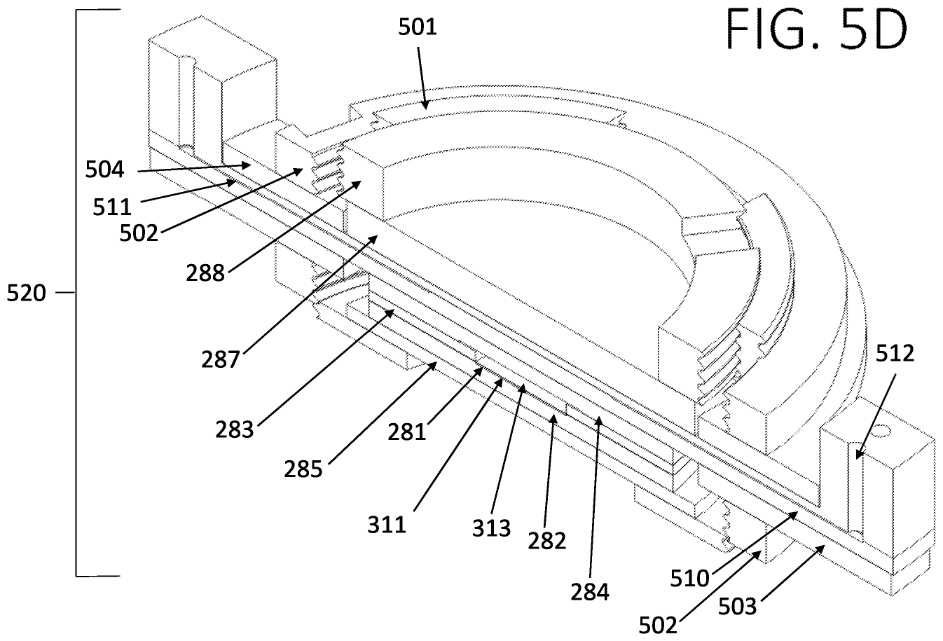

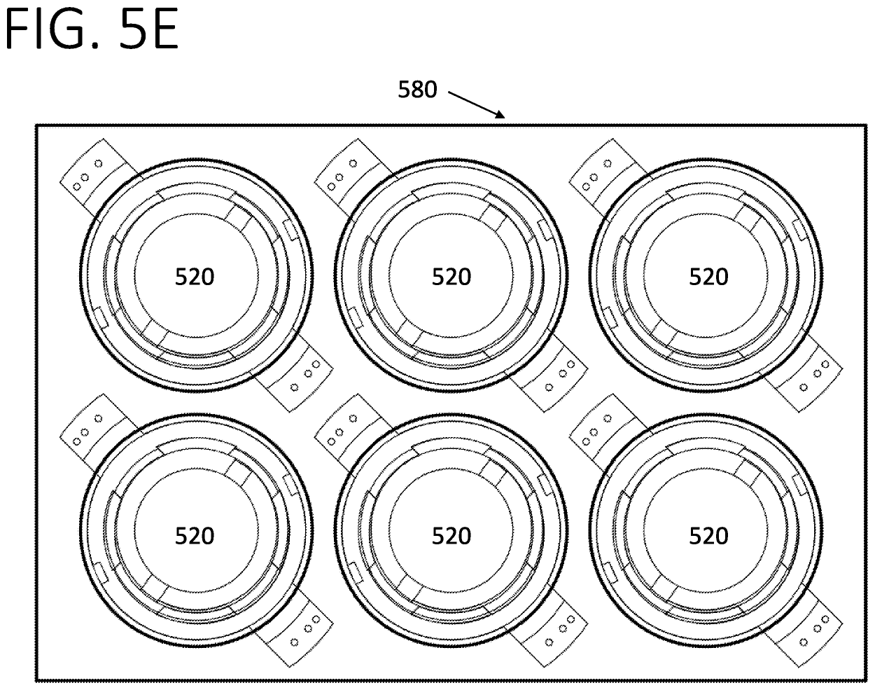

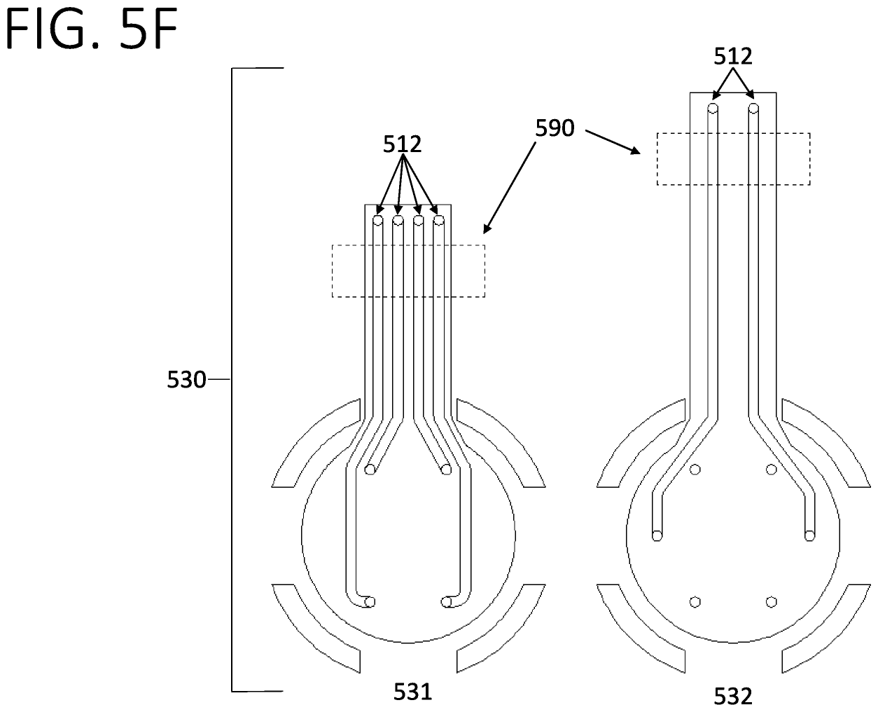

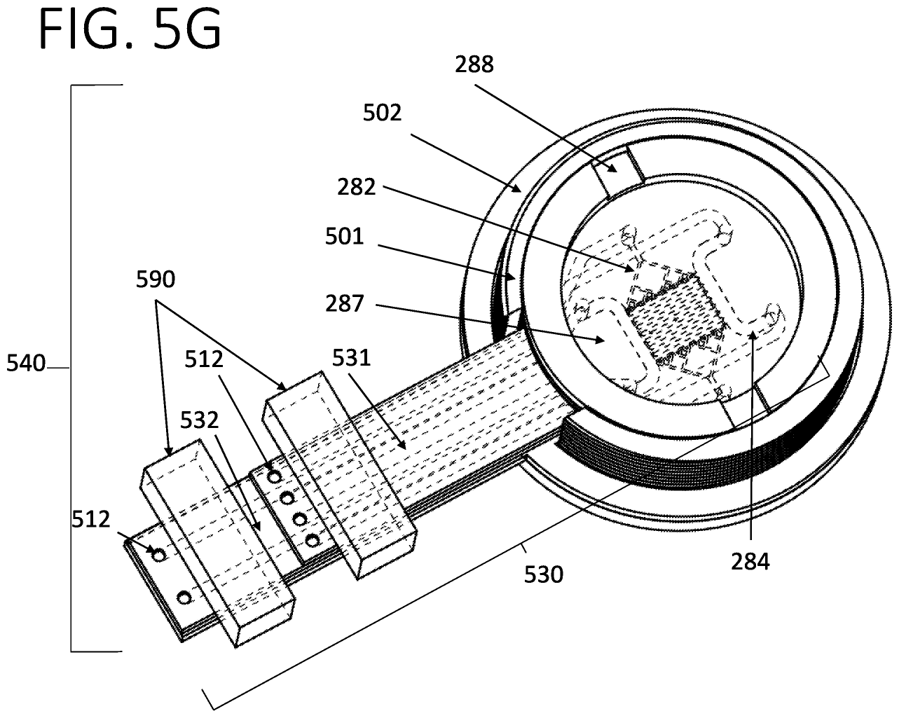

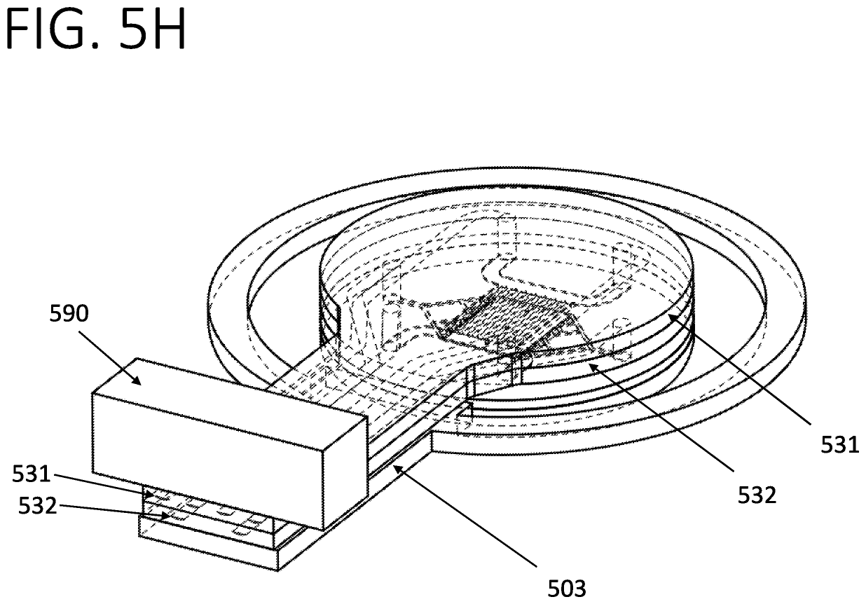

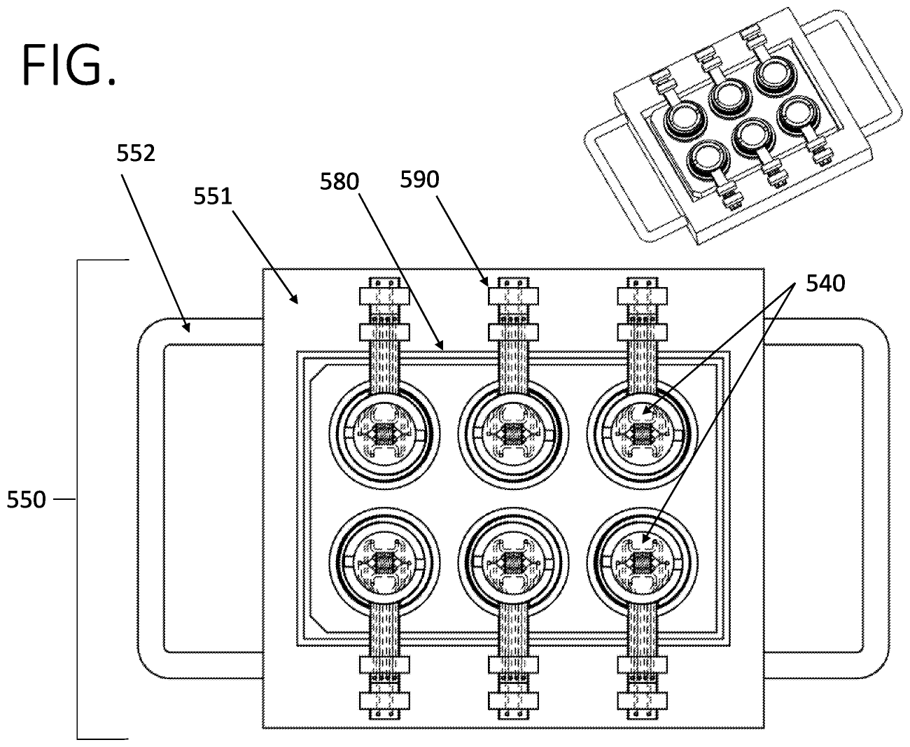

A limitation presented by each of these tubing implementations is that the insertion of additional chambers in the bioreactors requires that additional space be allocated for the tubing from lower layers to pass through upper ones. The tubing, guided by either holes or slots, also can limit the area of each layer available for fluidic channels within that layer. An alternative embodiment that addresses these problems involves having the fluidics from each layer exit the housing at that layer, which can be accomplished by cutting vertical slots in the tube as shown in FIG. 5A to create what can be described as a crown clamp.

Referring to FIG. 5A, the crown clamping device 500 includes an internally and externally threaded, notched crown housing 501, with one externally-threaded retaining ring 288, two internally threaded retaining rings 502, a fluidic interface bottom support 503, and a fluidic interface top support 504. The bottom supports are designed to provide mechanical support for the insertion of tubing or ribbon connectors into the fluidic and to protect the fluidic during handling of the device.

The two internally threaded rings 502 on the outside of the crown 501 may not be required depending upon the mechanical strength required of the notched crown housing 501 upon compression of the components within the crown by the externally threaded ring 288.

FIG. 5B is an isometric view of a microfluidic interface 510 with symmetric conduits 511 embossed on the underside, and vertical ports 512. In this embodiment, the two ports and channels furthest from the axis of symmetry are connected to the endothelial chamber of a two-chamber microfluidic bioreactor, and the four closest to the axis are for the stromal chamber of the reactor. In another embodiment, the ports and channels for one layer can be directed towards one slot in the crown, and the ports for the other layer to a different slot.

Referring to FIG. 5C, a two-chambered microfluidic bioreactor assembly 520 includes elements of the assembly 290 described in FIG. 2A, with the crown clamping device 500 and a microfluidic interface 510. Bioreactor 520 includes a microfluidic device whose layers can be pressed together to seal the compartments together, for example with a porous membrane 281 separating them, with the seals being created by compressing individual components, which can be fabricated by casting, embossing, injection molding, or machining, to form an integral unit.

FIGS. 5C and 5D show how the crown clamp 5000 in FIG. 5A is combined with the microfluidic interface 510 in FIG. 5B. Microfluidic interface 510 is compressed between the rigid pressure plate 287 and the microfluidic perfusion disc 284, bringing symmetric conduits 511 in contact with microfluidic ports on the perfusion disc 284. Membrane 281 is layered between an endothelial microfluidic disc 282 and a somatic cell chamber layer 283. The microfluidic perfusion disc 284 is mated to the top of the somatic cell chamber layer 283 to provide efficient nutrient delivery and waste removal to the somatic cells. This microfluidic assembly is then placed on a glass base 285 which fits inside the bioreactor housing 501 and rests on a bottom of the housing. The whole assembly is compressed by the upper threaded retaining ring 288, which is threaded into the bioreactor housing. Portions of fluidic interface 510 extending beyond the outer edge of bioreactor housing 501 are compressed between the fluidic interface bottom support 503 and the fluidic interface top support 504, which are secured in place with internally threaded retaining rings 502. Flexible lengths of the tubing (not shown for clarity) are then inserted into the vertical ports 512.

FIG. 5D provides a cross sectional view of a two-chambered assembly of a microfluidic bioreactor 520 including elements of the assembly 290 described in FIG. 2A with the crown clamping device 500 and the microfluidic interface 510. Bioreactor 520 includes a microfluidic device whose sealed chambers are created by compressing individual components, which can be fabricated by casting, embossing, injection molding, or machining, to form an integral unit. The microfluidic interface 510 is compressed between the rigid pressure plate 287 and the microfluidic perfusion disc 284, bringing the symmetric conduits 511 in contact with microfluidic ports on the perfusion disc 284. The membrane 281 is layered between the endothelial microfluidic disc 282 and the somatic cell chamber layer 283. The microfluidic perfusion disc 284 is mated to the top of the somatic cell chamber layer 283 to provide efficient nutrient delivery and waste removal to the somatic cells. This microfluidic assembly is then placed on a glass base 285 which fits inside the bioreactor housing 501 and rests on a bottom of the housing. The whole assembly is compressed by the upper, externally threaded retaining ring 288, which is threaded into the internally-threaded bioreactor housing. Portions of the fluidic interface 510 extending beyond the outer edge of bioreactor housing 501 are compressed between fluidic interface bottom support 503 and fluidic interface top support 504, which are secured in place with two internally threaded retaining rings 502. Flexible lengths of tubing (not shown for clarity) are then inserted into vertical ports 512. Two cell layers are formed in the bioreactor 520. The first is the endothelial cell layer 311, formed between the membrane 281 and the endothelial microfluidic disc 282. The second is a somatic cell layer 313 formed in the cell chamber layer 283 between the membrane 281 and the microfluidic perfusion disc 284.

As shown in FIG. 5E, a view of six (6) microfluidic bioreactors 520 arranged to fit in the footprint of a six-well cell culture plate (rectangular outline), the length of the fluidics outside of the clamping mechanism and the angle of the devices avoids interferences between adjacent bioreactors. This demonstrates the capability of multiplexing identical bioreactors for either high-throughput applications, for multiplexing otherwise identical bioreactors running under different flow conditions, or for connecting multiple bioreactors in series or parallel, as might be used to create a multi-organ microphysiological system.

FIG. 5F shows a microfluidic interface 530 with overlapping conduits 531 and 532 embossed on the underside, and vertical ports 512. Dotted lines show the approximate footprint of optional shut-off valves 590 according to some embodiments of the invention. The lengths of the two overlapping conduits 531 and 532 differ to avoid interferences between the two sets of vertical ports 512 or shut off valves 590.

FIG. 5G is a perspective view of a two-chambered embodiment of a microfluidic bioreactor 540 including elements of the assembly 290 (FIG. 2A) with the crown clamping device 500 and overlapping microfluidic interface 530. Note that both layers pass from the clamp housing through a common slot. Bioreactor 540 includes a microfluidic device that has multiple chambers that are sealed together by compressing individual components, which can be fabricated by casting, embossing, injection molding, or machining, to form an integral unit. The overlapping microfluidic interface 530 is compressed between the rigid pressure plate 287 and the microfluidic perfusion disc 284, bringing asymmetric conduits 531 and 532 into contact with microfluidic ports on the perfusion disc 284. The whole assembly is compressed by the upper threaded retaining ring 288 which is threaded into the bioreactor housing.

One embodiment of the single-slot format shown in FIGS. 5F and 5G has conduits 531 and 532 in the two layers that do not overlap vertically, as demonstrated by FIG. 5H, a perspective with cross-sectional detail of the fluidic interconnect for the microfluidic bioreactor 540. Portions of overlapping microfluidic conduits 531 and 532 extending beyond the outer edge of bioreactor housing 501 (not shown for clarity) are compressed between fluidic interface bottom support 503 and fluidic interface top support 504 (not shown for clarity). An optional shut-off valve 590 is placed so as to interrupt flow in microfluidic conduit 531. An additional shut-off valve can optionally be placed distal to the cross-section to interrupt flow in microfluidic conduit 532.

Referring to FIG. 5I, a multiplex microfluidic bioreactor array 550 includes a carrier 551 with handles 552 and six microfluidic bioreactors 540 as arranged to fit in the footprint of a standard six-well cell culture plate 580. This configuration demonstrates the capability of multiplexing identical bioreactors for high-throughput applications, for multiplexing otherwise identical bioreactors running under different flow conditions, or for connecting different organs in some combination of serial and parallel fluid flow as appropriate to match the physiology of a multiple-organ-on-a-chip microphysiological system. With proper placement of optional shut-off valves 590, each bioreactor's flow can be independently controlled, or synchronized with other bioreactors.

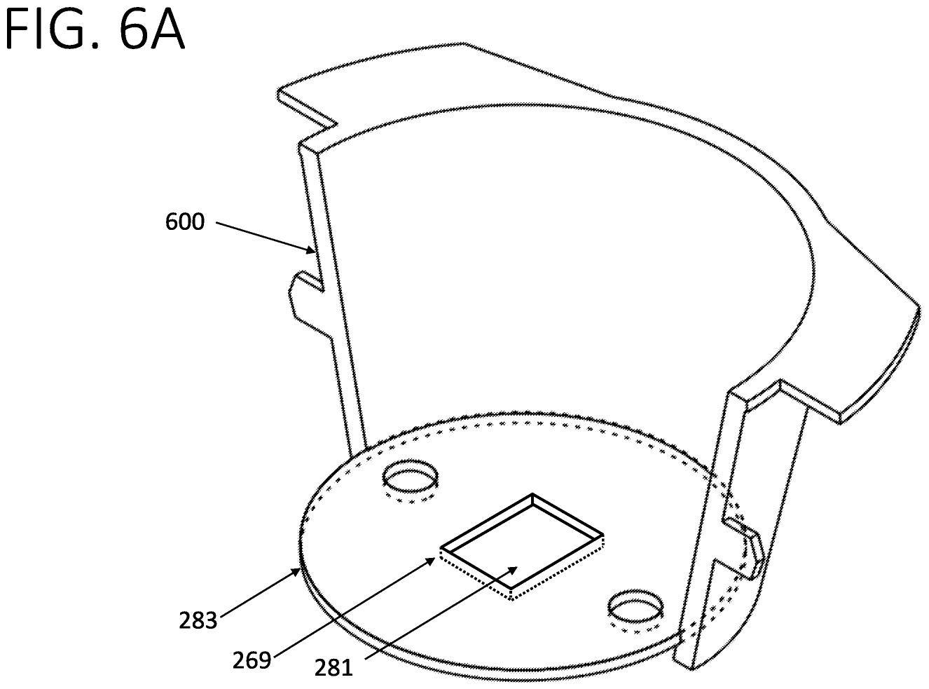

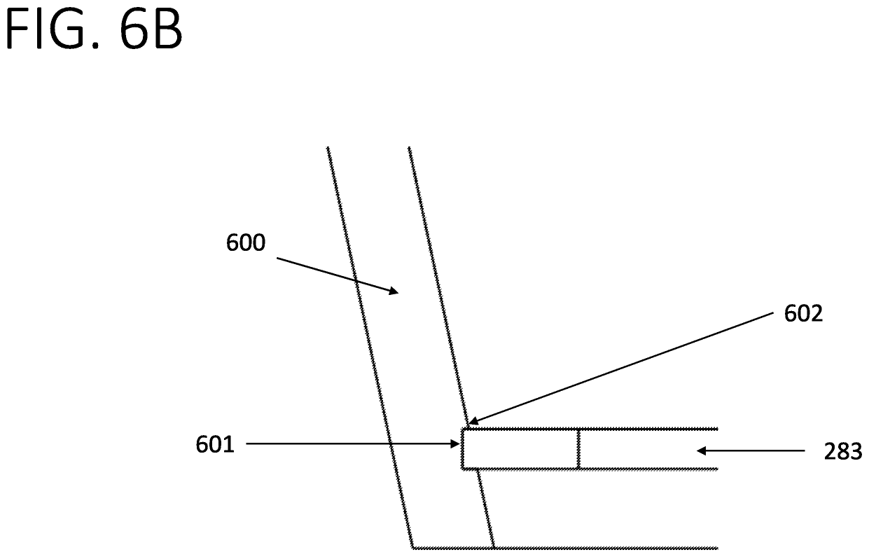

The perspective cutaway view of a transwell adapter 600 shown in FIG. 6A illustrates a disk-shaped somatic cell layer 283 with a cell-culture chamber 269 that is backed by membrane 281, snaps into the transwell adapter 600. This allows culture of cells independent of the bioreactor prior to insertion of, or after extraction of the somatic cell chamber layer 283 from any embodiment of the bioreactor.

Referring to FIG. 6B, a cross-section cutaway detail of the transwell adapter 600 shows how the somatic cell chamber disk 283 is supported by the transwell adapter 600. Internal groove 601 is cut into the inner surface of the transwell adapter 600, allowing the somatic cell chamber layer 283 to snap securely into place, captured by a small top retaining lip 602, and be later unsnapped and removed for either insertion into a bioreactor or for separate analyis.

In some applications of the microfluidic bioreactors, such as on the International Space Station with its microgravity environment, it is not acceptable to have fluid leak from the open ends of fluidic conduits that are open to the atmosphere. In many applications, it is not advisable to have microbial contamination enter the reactor through open ports, whether they are wet or dry, and hence there is a need for a means to sterilize recently made connections before fluid passes through them into an established cell culture chamber. Finally, when connecting a microfluidic bioreactor to its perfusion controller or to another bioreactor, it is imperative to eliminate air bubbles from these now-connected ports before fluid is passed through the conduits, which would thereby force any air bubbles in the fluidic connectors into the bioreactor chambers, where they could strip adherent cells from the sides or membranes of these chambers. In selected embodiments of this invention, we provide a valve mechanism that eliminates all of these problems.

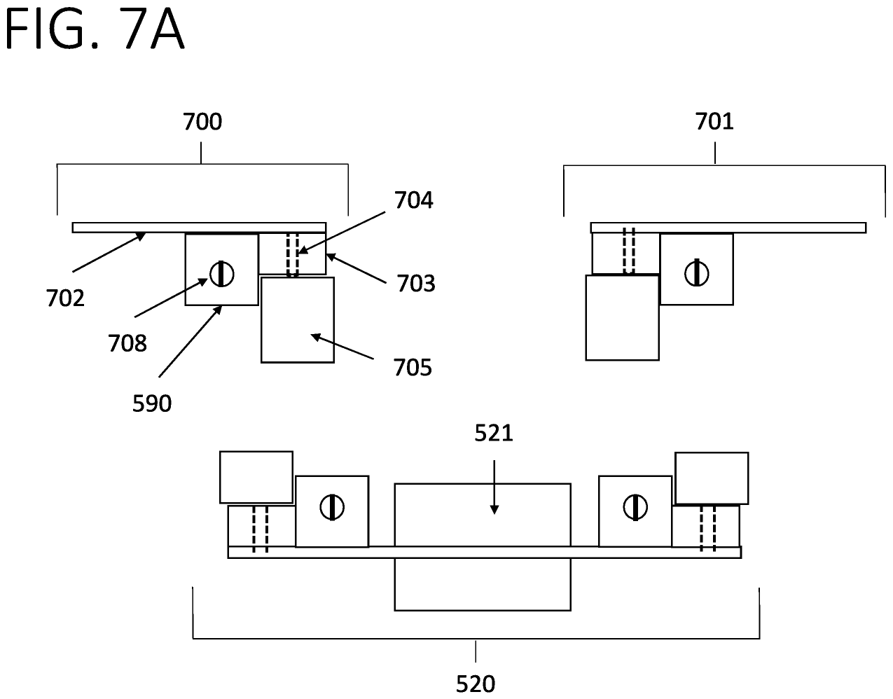

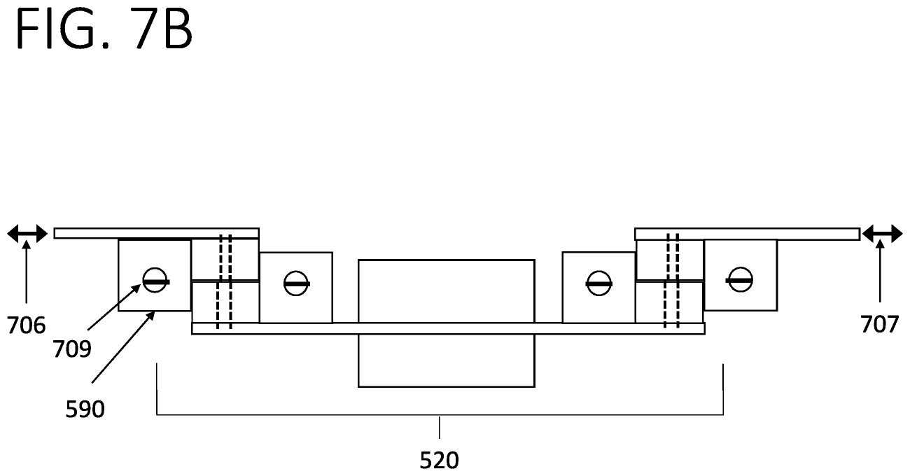

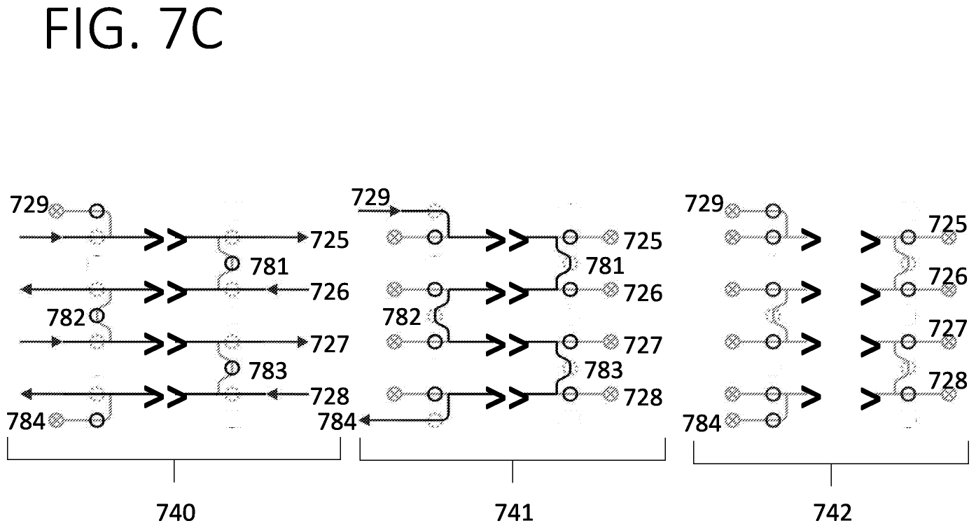

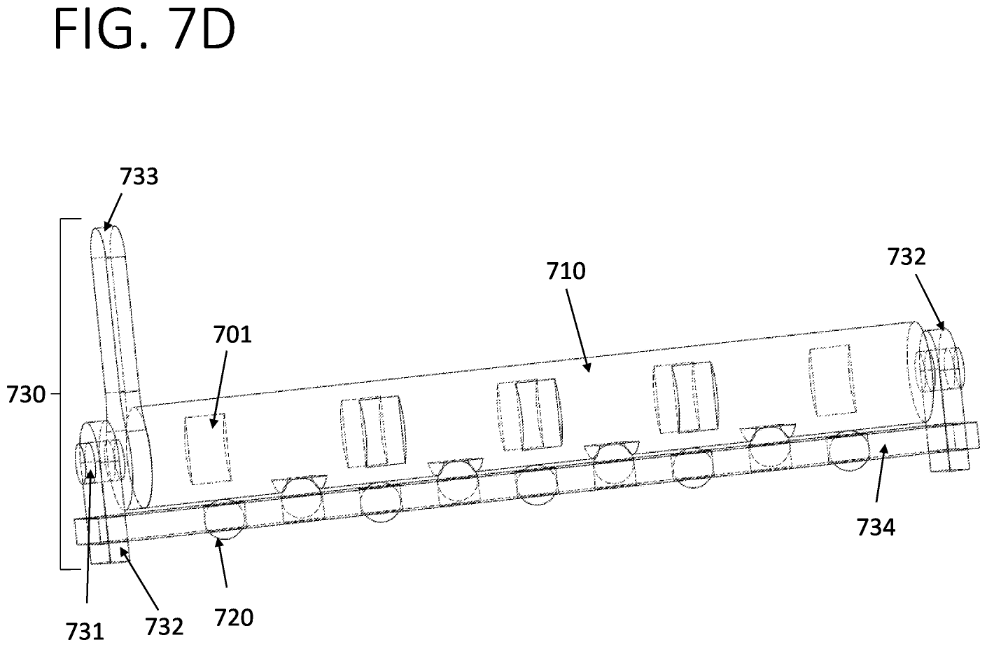

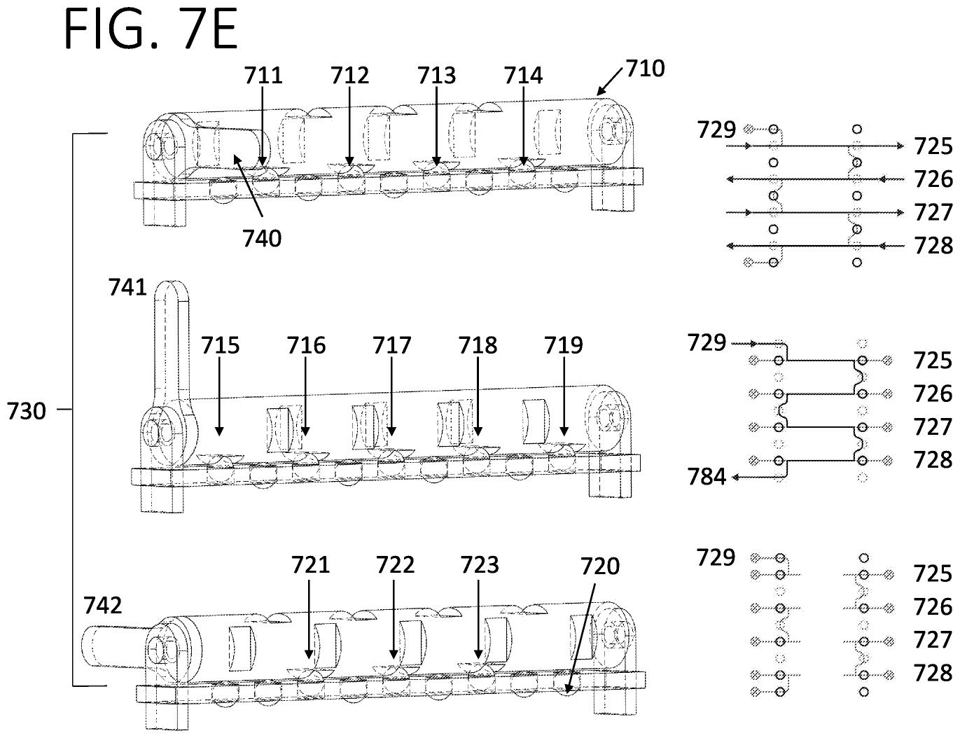

FIG. 7A shows a symmetric microfluidic bioreactor assembly 520 with bioreactor 521, a connector assembly 700 on the left, in this particular example connected to a perfusion controller, and a connector assembly 701 on the right that, in this particular example, would be connected to a second, downstream microfluidic bioreactor. A particular application of this architecture would be to have a liver-on-a-chip in bioreactor 521 and a brain-on-a-chip in the downstream bioreactor (not shown) that is at the other end of connector 701. In the configuration shown in FIG. 7A, all four cut-off valves 590 are in the off position 708 (the indicator bar in the circle is vertical) so that no fluid flows in any of the fluid conduits within any of the four fluid-carrying ribbons 702. Removable caps 705 seal the ports 704 in the port blocks 703 and the tubes (not shown) that are within the connector.

FIG. 7B shows the same connectors and bioreactors as in FIG. 7A, but in the operational, connected mode. The four caps 705 have been removed, the valves 590 have been switched to the on position 709 (the indicator bar in the circle is horizontal), and fluid 706 can flow to and from the perfusion controller on the left into the bioreactor 520. That bioreactor is connected to the downstream bioreactor, allowing fluid flows 707.

FIG. 7C presents a schematic showing the operation of the microfluidic valves compatible with various microfluidic interface devices (e.g., microfluidic interface 510, in one embodiment). Valves have three positions: Position A (“Run position”, 740), Position B (“Sterilize/Rinse/Dry/Remove Bubbles position”, 741), and Position C (“Disconnect position”, 742). In each position, the valve depresses some combination of flow restrictors into an underlying microfluidic conduit, selectively blocking or permitting flow as needed. In Position A (740), flow restrictors are in place to block rinse channel 729, while allowing flow in microfluidic channels 725, 726, 727, 728. Shunt channels 781, 782, and 783 and waste/vent channel 784 are all closed. In Position B (741), flow restrictors block microfluidic channels 725, 726, 727, 728 into or out of the bioreactor while allowing flow in rinse channel 729 to pass through shunt valves 781, 782, and 783 and then to waste/vent channel 784 to thereby sterilize and rinse all connector channels by delivering the appropriate solutions to 729 and removing them from 784; blow out fluid from all connectors by delivering pressurized gas to 729 and venting at 784, or by filling 729 with media until all bubbles trapped in the connectors are eliminated through 784. Note that in this mode, 729, 781, 782, 783, and 784 are all in series and enable the serial passage of fluid through every conduit or tube that will eventually allow fluid to flow, for example, from a perfusion controller on the left, as in 740, to an microfluidic bioreactor, for example, on the right. In Position C, flow restrictors block microfluidic channels 725, 726, 727, 728, 729, shunts 781, 782, and 783, and waste/vent 784, allowing disconnect of a microfluidic bioreactor from the system without introduction of extraneous flow, microbial contamination, or air bubbles into the bioreactor.

Referring to FIG. 7D, a perspective view of a microfluidic rotary cylindrical valve 730, the valve shaft 731 is either machined into each end or inserted through valve cam 710, which allows the assembly to rotate in the valve shaft supports 732. The valve selection lever 733 is affixed to the valve cam, and allows positioning of valve cam grooves 701 over valve-actuating balls 720, which are secured over microfluidic conduits (not shown) by ball retainer 734. Different angular positions of valve selection lever 733 permit different combinations of microfluidic conduits to be opened or closed, based on the pressure applied to the valve-actuating balls 720.

Referring to FIG. 7E, a schematic showing the operation of the microfluidic valve 730, valve cam 710 has three rotational positions: Position A (“Run position,” 740), Position B (“Steriize/Rinse/Dry/Remove Bubbles position,” 741), and Position C (“Disconnect position,” 742). In each rotational position, the valve cam 710 depresses some combination of valve-actuating balls 720 (collectively) into the underlying microfluidic conduits, selectively blocking or permitting flow as needed. In Position A (740), balls 720 are depressed by valve cam 710 to block rinse channel 729, while notches 711, 712, 713, and 714 leave balls 720 free which allows flow in microfluidic channels 725, 726, 727, 728 respectively. In Position B (741), balls 720 are depressed by valve cam 710 to block microfluidic channels 725, 726, 727, 728, while notches 715, 716, 717, 718 and 719 leave balls 720 free which allows flow in rinse channel 729. The same position supports removal of fluid or moisture in the channels by delivery of gas to 729 and venting at 729, and the removal of bubbles by delivering media to 729 and letting the bubbles escape through 784. In Position C (742), balls 720 are depressed by valve cam 710 to block microfluidic channels 725, 726, 727, 728, and 729, allowing disconnect of a microfluidic bioreactor from the system without introduction of extraneous flow or air bubbles into the bioreactor, and preventing leakage from the connection ports. Note that the exact tangential location of each of these recesses could be adjusted to be sure that hydraulic pressure was not applied to a bioreactor by the act of opening or closing its shut off valves.

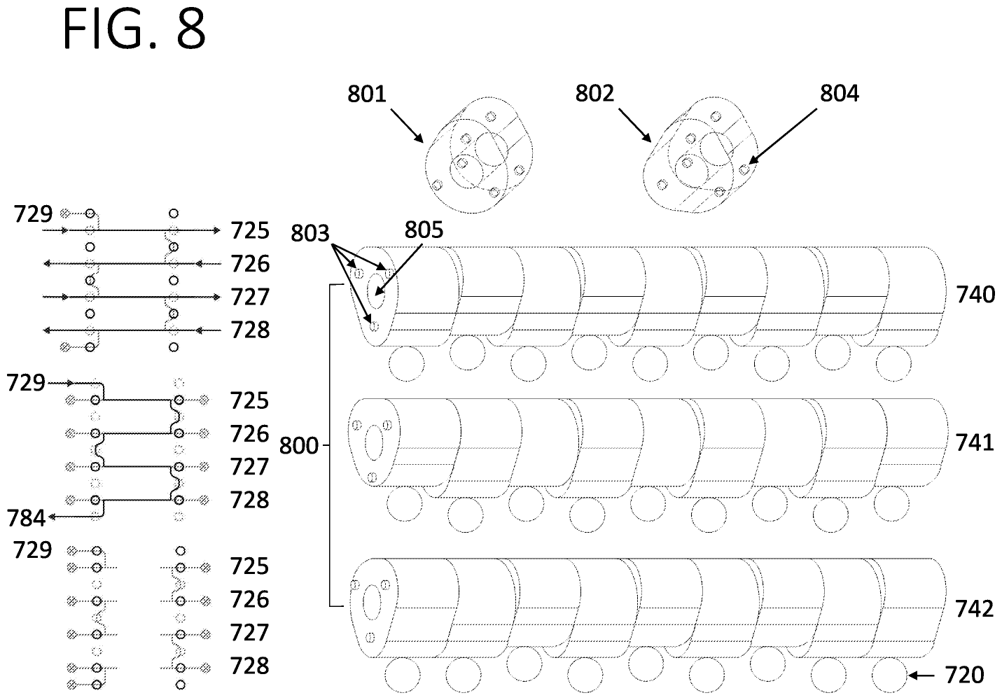

For other embodiments of cams for actuating the cylindrical rotary valve, referring to FIG. 8, a schematic showing the operation of the microfluidic valve cam 800 that is a configurable, modular valve cam, the modular valve cam 800 includes different combinations of a one-lobe cam 801 and two-lobe cam 802, with their relative angular alignment being set by protrusions 803 that mate with sockets 804 on the face of the adjacent cam. All cams are supported by a common cam shaft 805 that can, if desired, extend beyond the cams to provide mechanical support (not shown). In certain embodiments, modular valve cam 800 has 3 rotational positions: Position A (“Run position,” 740), Position B (“Sterilize/Rinse/Dry/Remove Bubbles position,” 741), and Position C (“Disconnect position,” 742). In each rotational position, modular valve cam 800 depresses some combination of valve-actuating balls 720 (collectively) into the immediatly underlying microfluidic conduits, selectively blocking or permitting flow as needed for each conduit. In Position A (740), balls 720 are depressed by modular valve cam 800 to block rinse channel 729, while notches leave balls 720 free which allows flow in microfluidic channels 725, 726, 727, 728. In Position B (741), balls 720 are depressed by modular valve cam 800 to block microfluidic channels 725, 726, 727, 728, while notches leave balls 720 free which allows flow in rinse channel 729. In Position C (742), balls 720 are depressed by modular valve cam 800 to block microfluidic channels 725, 726, 727, 728, and 729, allowing disconnect of a microfluidic bioreactor from the system without introduction of extraneous flow, microbial contamination, or air bubbles into the bioreactor. The same modes of operation discussed for FIG. 7E also apply in this embodiment.

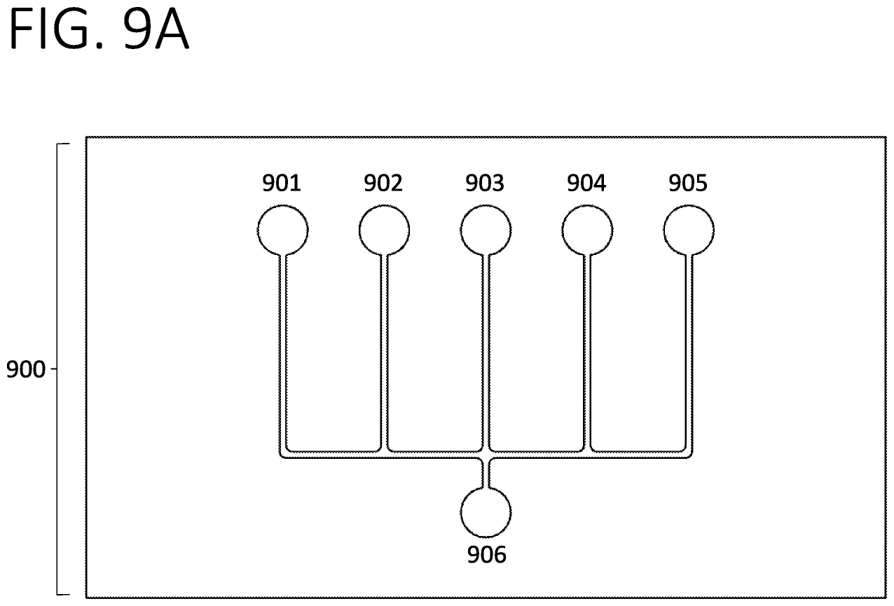

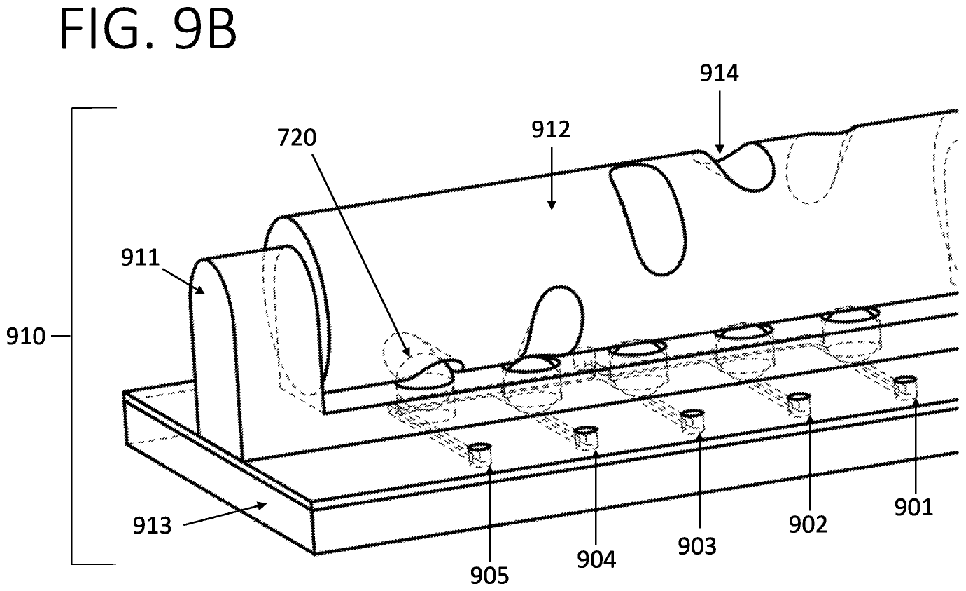

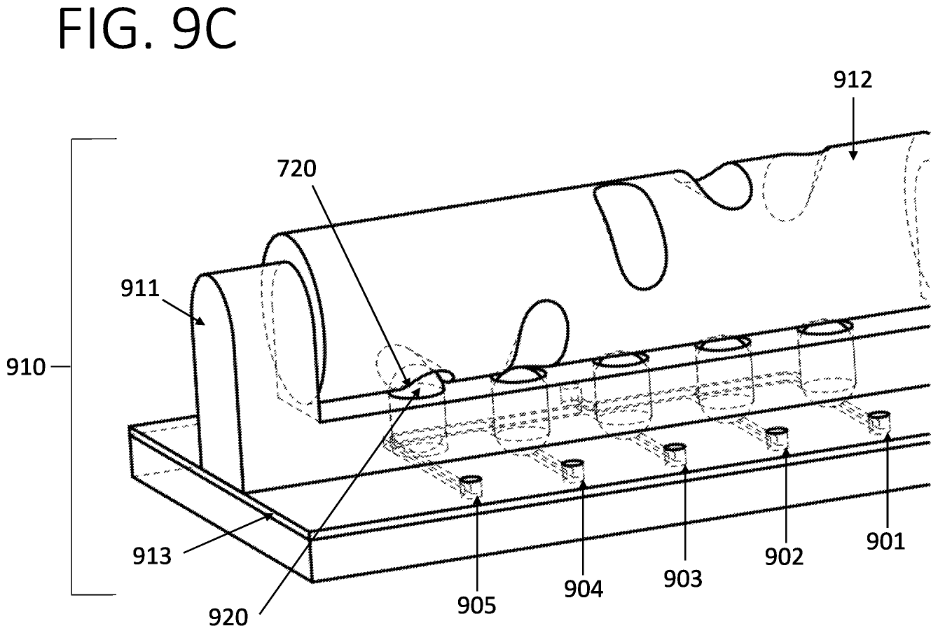

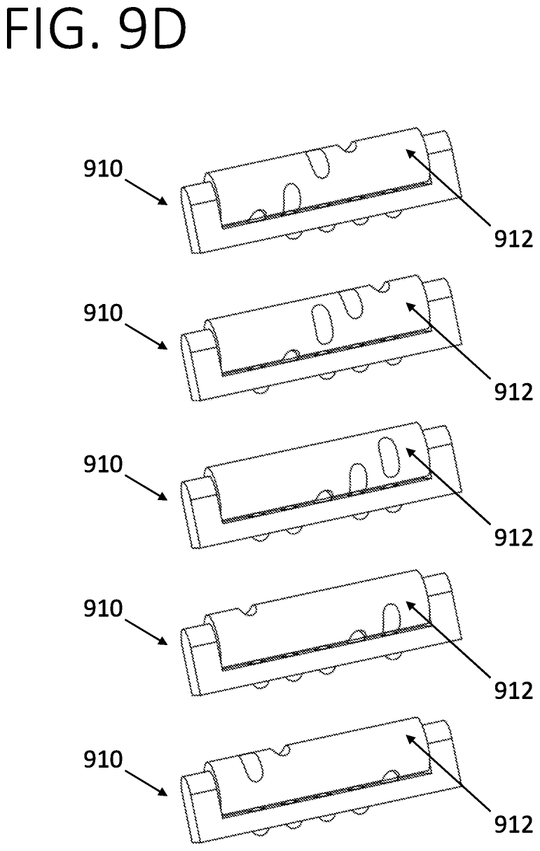

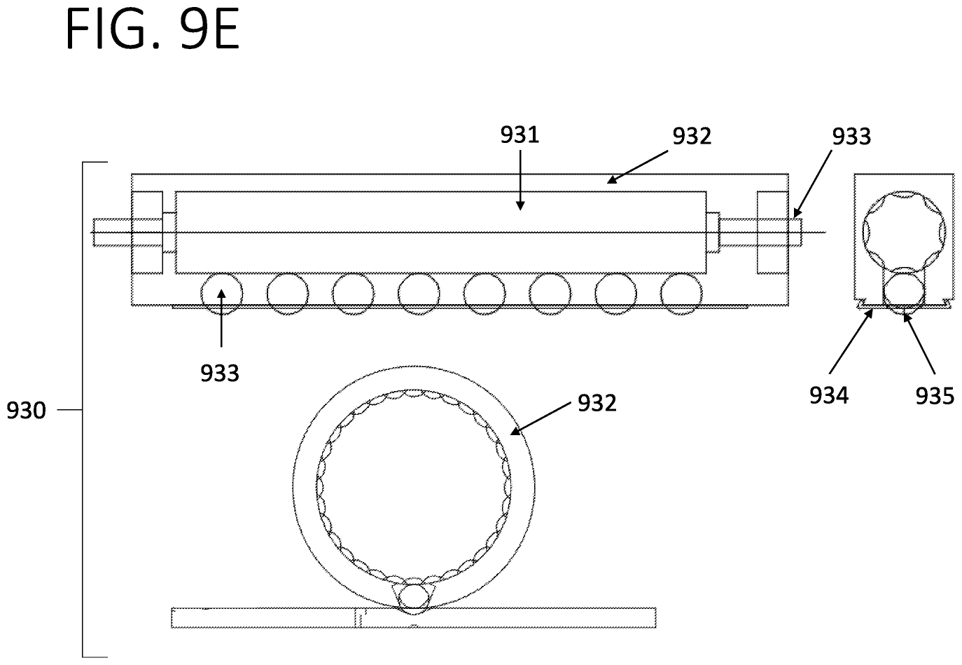

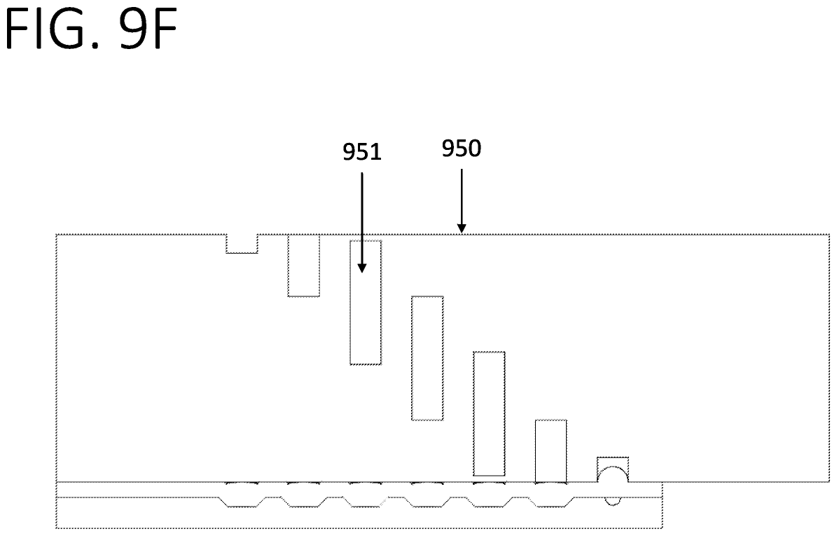

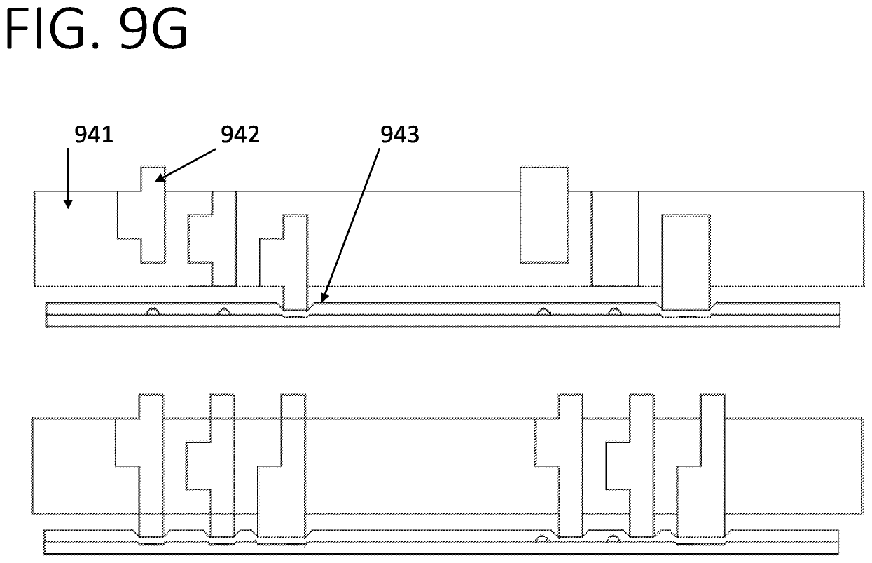

Referring to FIG. 9A, a plan-view schematic of the microfluidic manifold 900 for the rotating cylindrical valve includes five parallel ports 901-905, and a common port 906. In certain embodiments, different combinations of parallel ports can be connected with each other and the common port 906 by blocking flow from certain ports using rotary cylindrical valves whose actuators serve as flow restrictors. This type of manifold could be used, for example, to select which drug or nutrient solution would be delivered to the tubing ports 512 in FIG. 5C.

Referring to FIG. 9B, a perspective view of a spindle cam embodiment of a microfluidic rotating cylindrical valve 910, valve actuating ball flow restrictors 720 compress channel membrane 913 to close microfluidic channels under the valve-actuating balls 720. In certain embodiments, any input can be connected to any output by the proper placement of two recesses 914 in the spindle cam 912, or multiple inputs can be connected to a single output, or a single input can be connected to multiple outputs, etc. Additionally, by rotating the spindle cam 912 in spindle bracket 911, the cylindrical surface is in contact with the compressed valve-actuating balls, and the number of unique positions (and therefore number of actuated channels) can be increased to allow greater functionality in a smaller volume. The use of the rotating cylinder spindle cam allows the activation or deactivation of single or multiple channels in a specific, preprogrammed sequence.

Referring to FIG. 9C, a perspective view of a spindle cam embodiment of a microfluidic rotating cylindrical valve 910, cylindrical flow restrictors or actuating pins or rods with hemispherical ends 920 compress channel membrane 913 to close semi-cylindrical microfluidic channels under the cylinders 920. In certain embodiments, any input can be connected to any output by the proper placement of two recesses in the spindle cam 912, or multiple inputs can be connected to a single output, or a single input can be connected to multiple outputs, etc., as suggested by FIG. 9A. Additionally, by rotating the spindle cam 912 in spindle bracket 911, the cylindrical surface is in contact with the compressed valve actuating cylinders, and the number of unique positions (and therefore number of actuated channels) can be increased to allow greater functionality in a smaller volume. The use of the rotating cylinder spindle cam allows the activation or deactivation of single or multiple channels in a specific, preprogrammed sequence.

Referring to FIG. 9D, the rotating spindle cam 912 of the microfluidic rotating cylindrical valve 910 is rotated in steps to show the sequential compression or release of the cylindrical valve actuators as the recesses move to different angular positions.

Referring to FIG. 9E, the tumbler rotating cylindrical valve assembly 930 has a rotating cylinder 931 with drive shaft 933 captured in a housing 932 (in this embodiment cylindrical but other external shapes are possible) with a cylindrical interior cavity. That housing also holds tumbler balls 933 (or cylindrical pins, not shown) that are held in place by a retainer clip 934 that allows the balls to protrude from (935) but not escape from the tumbler body.

Referring to FIG. 9F, the recesses 951 in the rotating cylindrical actuator of the rotary cylindrical valve 950 can be staggered to enable sequential opening and closing of valves, which could be used to provide pumping functions.

Referring to FIG. 9G, the rotating cylinder 941 can drive vertically constrained actuators 942 that compress the fluidic circuit 943. Pins in the rotating cylinder (941) can drive the actuators. The actuators can be of a variety of widths to control the number of channels being opened or closed, as illustrated by the different embodiments shown.

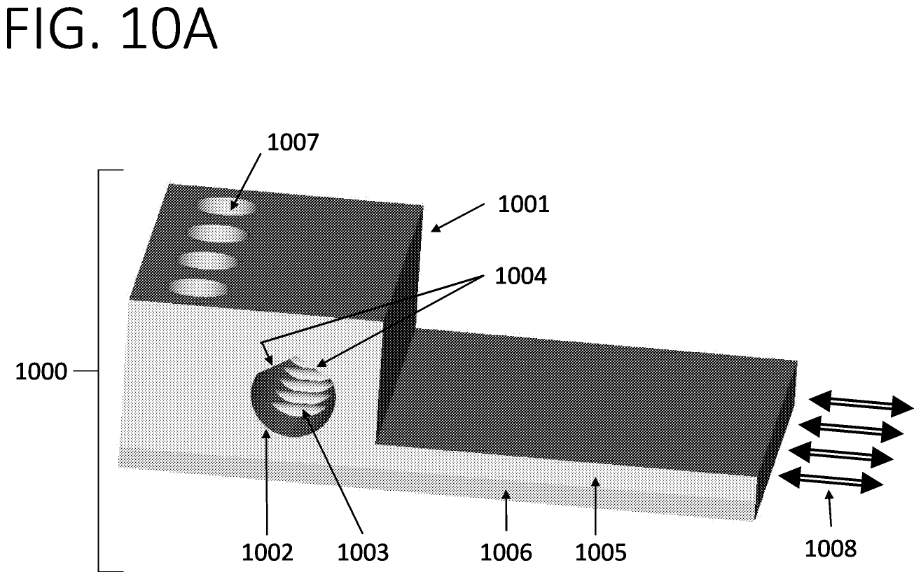

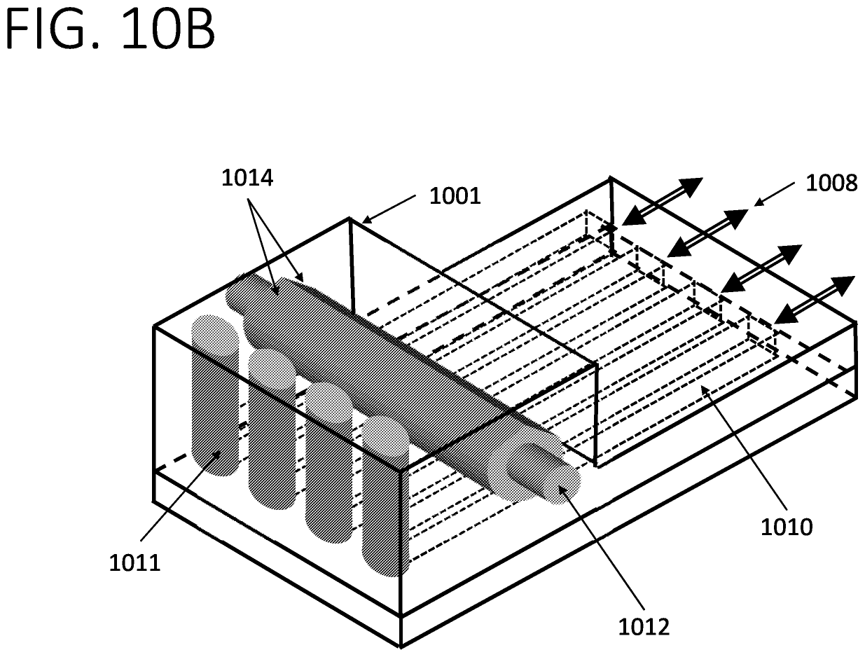

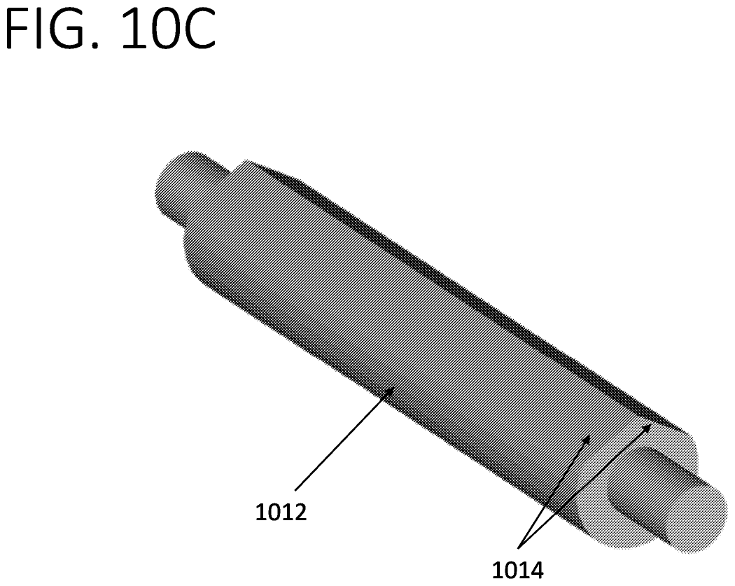

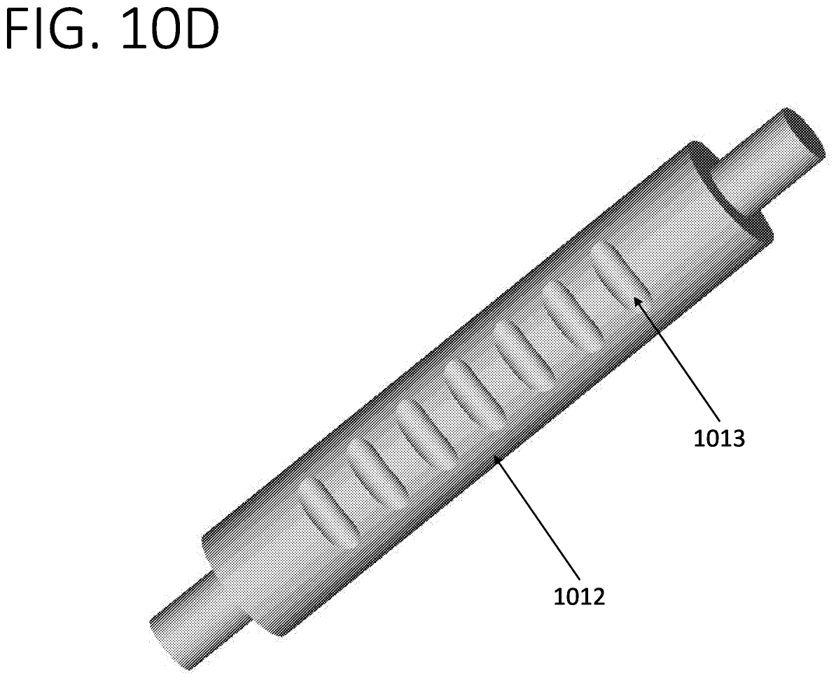

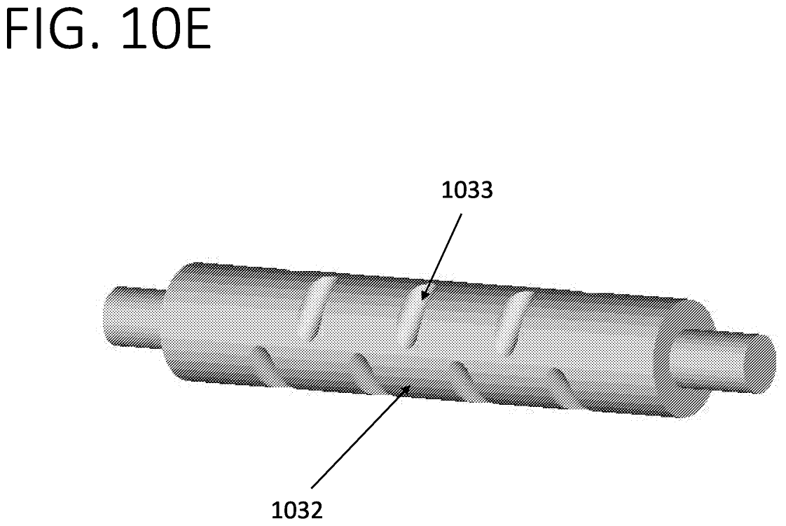

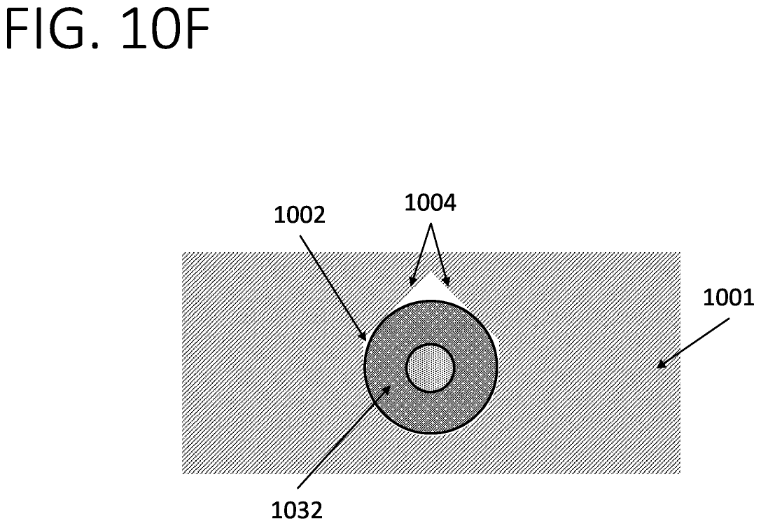

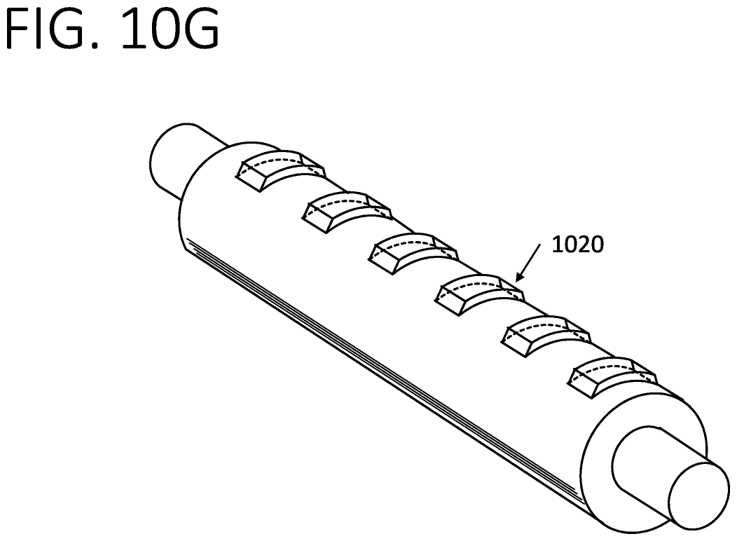

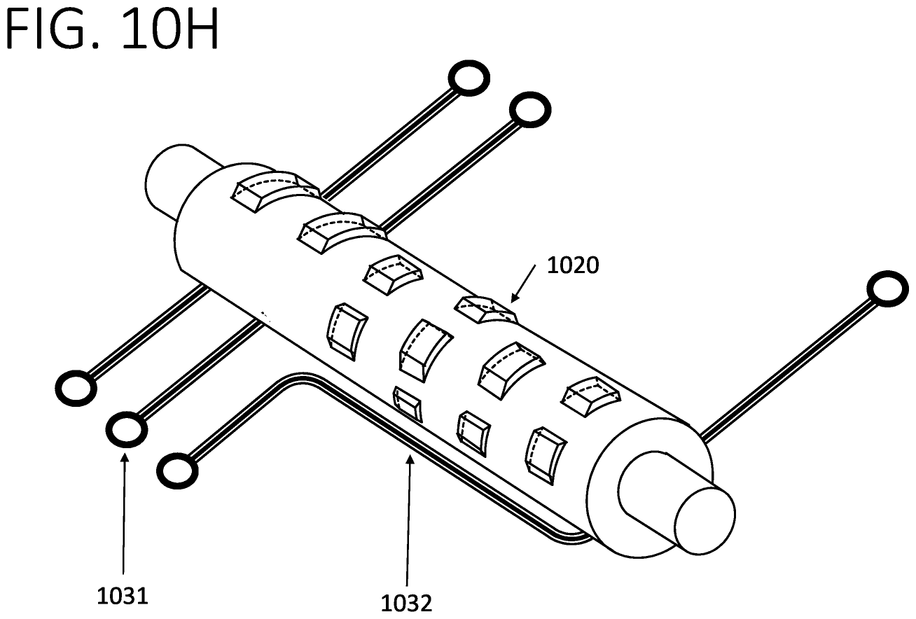

Referring to FIG. 10A, the end of the microfluidic interconnect system 1000 that was discussed in detail above can have an integrally cast valve body 1001 with a molded cavity 1002 in which the rotary cylindrical valve actuator operates. Within the cavity, there are raised regions 1003 beneath which the fluidic conduits are located. These conduits connect the ports 1007 to the internal conduits that carry fluids 1008 to the connected microfluidic bioreactor, perfusion controller, or other instrument. Planar surfaces 1004 at the top of the cavity maintain the alignment of the cylindrical valve actuator as it rotates and delivers compressive forces to the conduits underneath 1003. The conduits are formed in either the lower surface 1005 of the valve body or the upper surface of the membrane 1006 that seals the conduits. The key feature of this approach is that the valve has only two parts—the valve body that integral to the microfluidic interconnect, and the single-piece valve body. In one embodiment, both can be cast or injection molded, the former from a flexible elastomer and the latter from a rigid material. This greatly simplifies the fabrication and reduces the cost of the cut-off valve that would be part of the bioreactor fluidics discussed above.

Referring to FIG. 10B, we see that using a machined casting form 1012 allows tension and compression zones to be specifically engineered to meet the varying needs of different fluidic designs. The top machined surface allows the compression tension to be adjusted to accommodate different numbers of valves. Using two angled planar surfaces 1014 helps to keep the actuator centered when turning in either direction. The bottom surface of the casting form defines the compression ridges and eliminates the need for an intermediate actuator like a ball or pin. When the machined actuator is inserted into the cast hole, the entire mechanism is fully functional with no other mechanical support required. This allows valves to be constructed at the ends of long fluidic ribbon cables without the need for rigid supports or clamping structures.

Referring again to FIG. 10B, the position of the internal conduits 1010 relative to the valve body is evident. The drawing shows the four casting pins 1011 for the ports and the casting mold 1012 for the actuator region still in place. When the casting pins 1011 and the casting mold 1012 are removed, the resulting shape is the cavity, as shown in FIG. 10A.

Referring to FIG. 10C, the upper surface of the casting mold 1012 for the integrally cast rotary cylindrical valve shows how the planar surfaces 1014 create the planar surfaces 1004 in the cavity 1002 shown in FIG. 10A.

Referring to FIG. 10D, one can see how the lower surface of the casting mold 1012 for the integrally cast rotary cylindrical valve with grooves 1013 creates the raised regions 1003 above the conduits in FIG. 10A. These regions ensure efficient and complete sealing of the microfluidic conduits beneath 1003.

FIG. 10E shows the cylindrical actuator 1032 with compression-release grooves 1033 for the integrally cast rotary cylindrical valve. The angular positioning of the recesses in this actuator 1030 determine at what angle each conduit will be open or closed.

FIG. 10F shows an end view that demonstrates how the cylindrical actuator 1032 fits into the cavity 1002 in the valve body 1001 and is kept centered by the planar surfaces 1004. The raised regions inside of 1002 and the fluidic conduits are not visible from the outside.

Referring to FIG. 10G shows a normally-open rotating cylindrical valve with elevated actuating surfaces 1020 rather than recessed ones. In this case, there is no need for the raised regions 1003 in the cavity 1002 in FIG. 10A.

Referring to FIG. 10H, a rotating cylindrical valve with elevated actuating surfaces can also provide pumping functions by sequential compression of a longitudinal fluidic conduit 1032. Conduits 1031 are either open or closed depending upon the angular position of the raised actuators 1020. Not shown, but the actuator could have a combination of raised and recessed regions to perform different valving and pumping functions, some of which are determined by the raised regions 1003 in FIG. 10A, and others chosen whether or not the actuator has a particular raised region 1020 above a fluidic conduit that does not have a corresponding raised region.

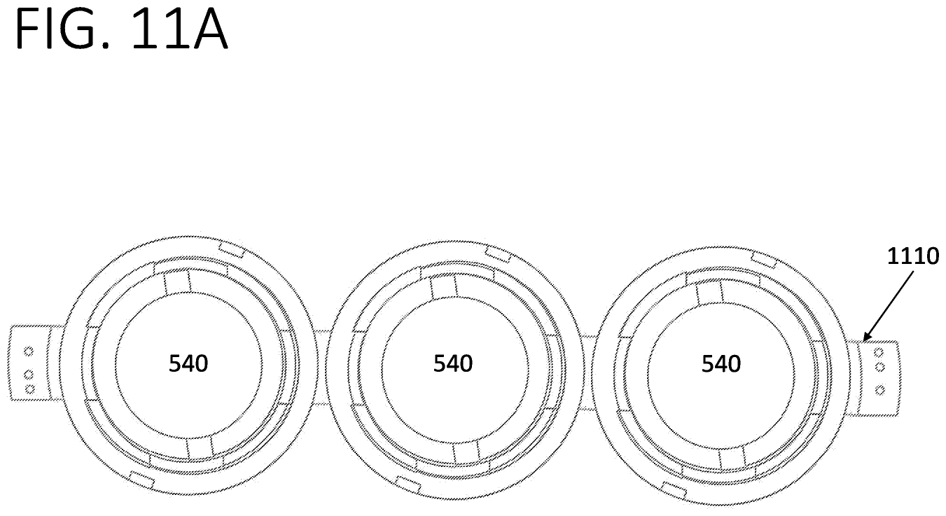

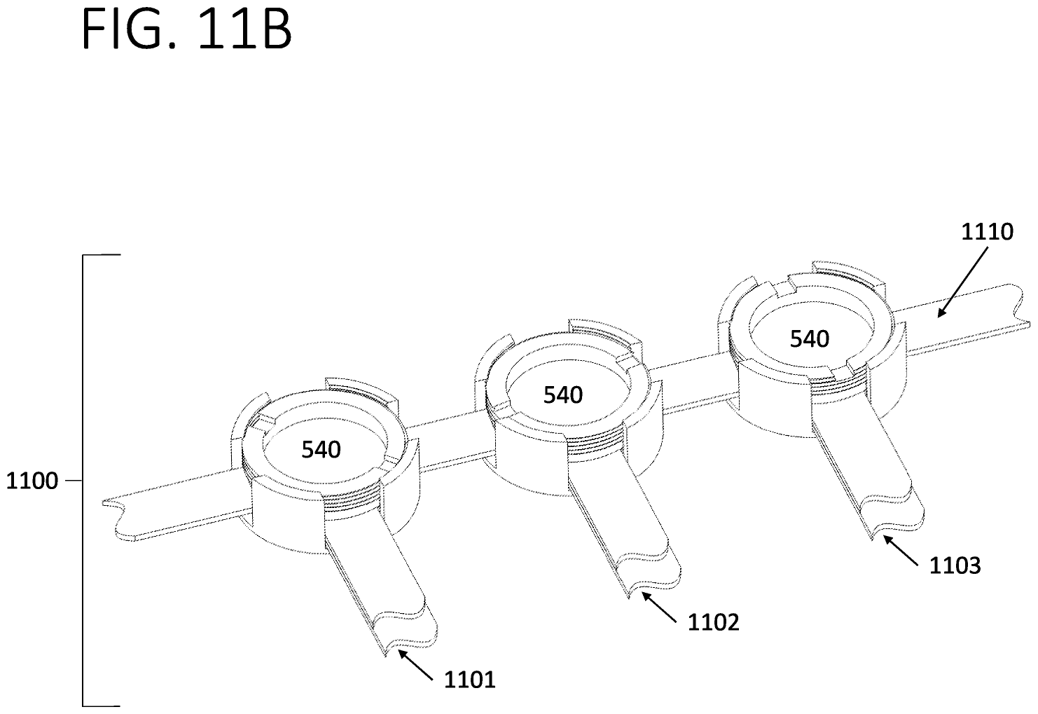

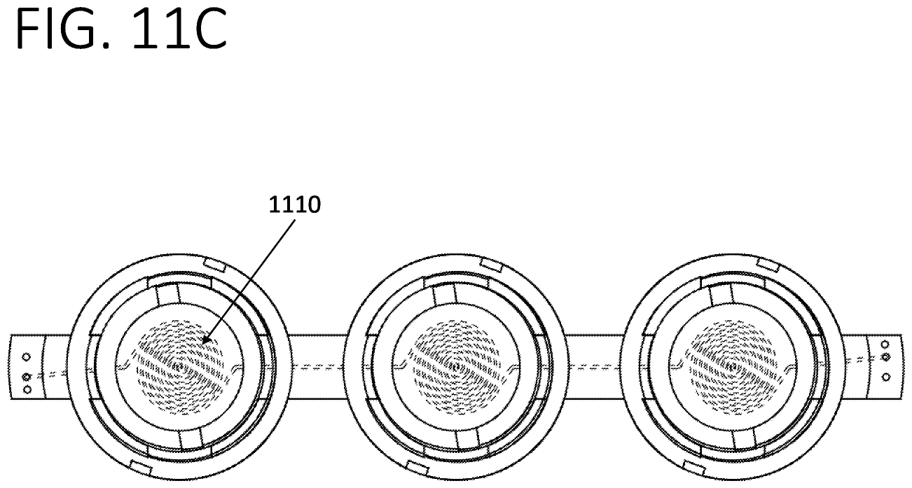

FIG. 11A shows a plan schematic of three symmetrical crown-clamp bioreactors housings 540 with serially connected bioreactors. In the simplest embodiment, all fluidics layers would be common to all three reactors, but it would be possible to have separate delivery conduits run the length of the common fluidic 1110.

FIG. 11B shows a perspective view of a three-element bioreactor chain 1100 according to one embodiment of the invention. The key feature of this embodiment is that the common layer 1110 of the bioreactors can be assigned to any of the layers in the three individual bioreactors 540. If 1110 is an in-series endothelial microfluidic strip, three 3-layer microfluidic bioreactors 540 therefore share common flow for endothelial cells, but not for somatic cells, and perfusion controllers connected at 1101, 1102, or 1103 can provide or remove different media to each of 540. Such system could model the zonation of the liver, since distal regions of the liver sinusoid operate with differing nutrient, metabolite, and pH levels. Alternatively, 1110 could represent the lumen of the gastrointestinal track, and 1101, 1102, and 1103 could provide differing levels of oxygen, bile salts, or fluids to represent different regions of the gastrointestinal track.

Referring to FIG. 11C, it is clear that the design can readily implement the daisy-chain bioreactor developed by Shah et al., according to their modular, microfluidics-based, human—microbial crosstalk (HuMiX) bioreactor [1], without requiring the large number of screws to seal the layered HuMiX bioreactor.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

### LISTING OF REFERENCES

[1]. Shah et al., Nature Communications, “A microfluidics-based in vitro model of the gastrointestinal human-microbe interface,” 7:11535, 2016.
[2]. Brunswig, et al., Content modification control using read-only type definitions. U.S. Pat. No. 8,533,413, Sep. 10, 2013.
[3]. Wikswo et al., Engineering Challenges for Instrumenting and Controlling Integrated Organ-on-Chip Systems. IEEE Trans. Biomed. Eng., 60:682-690, 2013.
[4]. Wikswo et al., Scaling and systems biology for integrating multiple organs-on-a-chip. Lab Chip, 13:3496-3511, 2013.
[5]. Block, III, et al., Normally closed microvalve and applications of the same, U.S. Pat. No. 9,618,129, Apr. 11, 2017.
[6]. Unger et al., Monolithic microfabricated valves and pumps by multilayer soft lithography. Science, 288:113-116, 2000.
[7]. Grover et al., Monolithic membrane valves and diaphragm pumps for practical large-scale integration into glass microfluidic devices. Sensors and Actuators B-Chemical, 89:315-323, 2003.
[8]. Browne et al., “A PDMS pinch-valve module embedded in rigid polymer lab chips for on-chip flow regulation,” J. Micromech. Microeng., 19:115012, 2009.
[9]. Loth et al., “Microfluidic High Speed Pinch Valve,” Proceedings of the 10th IEEE International Conference on Nano/Micro Engineered and Molecular Systems (IEEE-NEMS 2015) pp. 9-14.

1. A multichamber bioreactor, comprising:
multiple planar layers stacked on each other defining at least one chamber; and
a clamping mechanism, wherein the clamping mechanism comprises a housing and retaining means received in the housing and configured to generate a controlled and uniform pressure to secure the stacked multiple planar layers in the housing.

2. (canceled)

3. The multichamber bioreactor of claim 1, wherein the stacked multiple planar layers comprise:
an endothelial microfluidic disc;
at least one somatic cell chamber layer disposed on the endothelial microfluidic disc;
a microfluidic perfusion disc disposed on the at least one somatic cell chamber layer;
a pressure plate disposed on the at least one somatic cell chamber layer; and
at least one membrane, each member disposed between the endothelial microfluidic disc and the at least one somatic cell chamber layer, and between two adjacent somatic cell chamber layers when the at least one somatic cell chamber layer has two or more somatic cell chamber layer,
wherein the stacked multiple planar layers are placed on a base plate, received in the housing.

4. The multichamber bioreactor of claim 3, wherein each of the pressure plate and the microfluidic perfusion disc has a plurality of through holes defined therein, and aligned to each other in the stacked multiple planar layers, such that a plurality of tubes with flexible lengths is insertable into the through holes of the pressure plate and the microfluidic perfusion disc for connecting to an individual layer.

5. The multichamber bioreactor of claim 3, wherein
the microfluidic perfusion disc has notches formed on its edge and tubing sockets protruded from the microfluidic perfusion disc for connecting a plurality of tubes to an individual layer;
the somatic cell chamber layer has tubing sockets protruded from the somatic cell chamber layer for connecting the plurality of tubes to an individual layer; and
the pressure plate has notches formed its edge, such that, as assembled, the tubing sockets of the somatic cell chamber layer pass through the notches of the microfluidic perfusion disc, wherein both sets of sockets are in turn received in the notches of the pressure plate, so as to allow each upper layer to be inserted over a lower layer without having to thread the plurality of tubes through individual holes in each upper layer.

6. The multichamber bioreactor of claim 3, further comprising a transwell adapter for accommodating the at least one somatic cell chamber layer to allow culture of cells independent of the bioreactor prior to insertion of, or after extraction of the at least one somatic cell chamber layer.

7-10. (canceled)

11. The multichamber bioreactor of claim 1, wherein the housing is an internally threaded, notched crown housing, and wherein the retaining means comprises an externally threaded ring; a fluidic interface bottom support; a fluidic interface top support; and a microfluidic interface disposed between the fluidic interface bottom support and the fluidic interface top support, wherein the fluidic interface bottom and top supports are designed to provide mechanical support for insertion of tubes or ribbon connectors into fluidic and to protect the fluidic during handling of the multichamber bioreactor.

12. The multichamber bioreactor of claim 11, wherein the microfluidic interface comprises conduits embossed on underside and vertical ports.

13. The multichamber bioreactor of claim 12, wherein the two ports and channels furthest from an axis of the conduits are connected to an endothelial chamber of the endothelial microfluidic disc, and the four ports and channels closest to the axis are connected to a stromal cell chamber of the somatic cell chamber layer.

14. The multichamber bioreactor of claim 12, wherein the ports and channels for one layer are directed towards one slot of the crown housing, and the ports for the other layer are directed to a different slot of the crown housing.

15. The multichamber bioreactor of claim 12, wherein the microfluidic interface further comprises shut-off valves coupled between the conduits and the ports.

16-21. (canceled)

22. A clamping device, comprising:
a housing; and
retaining means received in the housing and configured to generate a controlled and uniform pressure to secure a layered, planar multi-chamber microfluidic bioreactor in the housing.

23. The clamping device of claim 22, wherein the housing has a threaded inner surface, wherein the retaining means comprises at least one threaded retaining ring being operably threaded into the housing to secure the layered, planar multi-chamber microfluidic bioreactor in the housing.

24. The clamping device of claim 22, wherein the housing has slots formed in a wall of the housing, and wherein the retaining means comprises a retaining ring having pins radially protruded from a peripheral side of the retaining ring being operably fitted into the slots of the housing to secure the layered, planar multi-chamber microfluidic bioreactor in the housing.

25. The clamping device of claim 24, wherein the slots are L-shape slots.

26. The clamping device of claim 22, wherein the retaining means comprises an expanding clamp pressing outwards against an inner surface of the housing to be held in place by force of friction between the sides of the expanding clamp and the inner surface of the housing.

27. The clamping device of claim 22, wherein the housing includes an internally threaded, notched crown housing, and wherein the retaining means comprises an externally-threaded retaining ring that fits inside the crown housing.

28-35. (canceled)

36. A rotating cylindrical valve, comprising:
actuators having elevated actuating surfaces for providing pumping functions by sequential compression of a longitudinal fluidic conduit.

37. The rotating cylindrical valve of claim 36, wherein the conduit is either opened or closed depending upon an angular position of the actuators.

38. The rotating cylindrical valve of claim 36, wherein the actuators have a combination of raised and/or recessed regions to enable sequential opening and closing of valves as so to perform different valving and pumping functions.

39. The rotating cylindrical valve of claim 36, wherein the actuators have a variety of widths to control the number of channels being opened or closed.

39-47. (canceled)
